SEQUENCE LISTING (15 sequences)

SEQ ID NO: 1 - DNA, 1635 bp, synthetic construct
  source 1..1635: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 2 - DNA, 1512 bp, synthetic construct
  source 1..1512: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtcaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 3 - DNA, 1509 bp, synthetic construct
  source 1..1509: mol_type = other DNA
atggctctgcctgtgacagctctgctgctgcctctggctctgcttctgcatgccgccagacctgacgtggtcatgacacagacacctctgagcctgcctgtgtctctgggagatcaggccagcatcagctgcagatctagccagagcctggtgcacagcaacggcaacacctacctgcggtggtatctgcagaagcccggccagtctcctaaggtgctgatctacaaggtgtccaacagagtgtccggcgtgcccgatagattttctggcagcggctctggcaccgacttcaccctgaagatcaatagagtggaagccgaggacctgggcgtgtacttctgtagccagtctacccacgtgccatggacctttggcggcggaacaaagctggaaatcaagagcagcgccgacgacgccaagaaggacgccgctaagaaggatgacgccaaaaaagacgatgccaaaaaggatggcggcgtgaagctggacgaaacaggcggaggacttgttcagcctggcggagccatgaagctgagctgtgtgaccagcggcttcaccttcggccactactggatgaactgggtccgacagagccctgagaaaggcctggaatgggtcgcccagttcagaaacaagccctacaactacgaaacctactacagcgacagcgtgaagggcagattcaccatcagccgggacgacagcaagtccagcgtgtacctgcagatgaacaacctgcgcgtggaagataccggcatctactactgtaccggcgccagctacggcatggaatatctcggccagggcaccagcgtgaccgtgtctacaacaacccctgctcctcggcctcctacaccagctcctacaattgccagccagccactgtctctgaggcccgaagcttgtagacctgctgcaggcggagccgtgcatacaagaggactggatttcgcctgcgacttctgggtgctcgtggttgttggcggagtgctggcttgttactccctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagatccagagtgaagttcagcagatccgccgacgctcctgcctatcagcagggacagaaccagctgtacaacgagctgaacctggggagaagagaagagtacgacgtgctggacaagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgccacctagatgatga

SEQ ID NO: 4 - protein, 254 aa, synthetic construct
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVS

SEQ ID NO: 5 - protein, 21 aa, synthetic construct
MALPVTALLLPLALLLHAARP

SEQ ID NO: 6 - protein, 45 aa, synthetic construct
TTTPAPRPPTPAPTIASQPLSLRPEACRPAAGGAVHTRGLDFACD

SEQ ID NO: 7 - protein, 27 aa, synthetic construct
FWVLVVVGGVLACYSLLVTVAFIIFWV

SEQ ID NO: 8 - protein, 41 aa, synthetic construct
RSKRSRLLHSDYMNMTPRRPGPTRKHYQPYAPPRDFAAYRS

SEQ ID NO: 9 - protein, 42 aa, synthetic construct
KRGRKKLLYIFKQPFMRPVQTTQEEDGCSCRFPEEEEGGCEL

SEQ ID NO: 10 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 11 - protein, 543 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 12 - protein, 502 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 13 - protein, 501 aa, synthetic construct
MALPVTALLLPLALLLHAARPDVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 14 - protein, 249 aa, synthetic construct
QVRLQGSGPSLVKPSQTLSLTCTVSGFSLTSNAVDWVRQAPGKVPEWLGFIRGGGSTFYNSALKSRLSITRDTSKSQVSLSLSSVTTEDTAVYYCARASCSGDIYTDTCGIDYWGPGLLVTVSSEGKSSGSGSESKVDQSALTQPSSVSRSLGQSVSITCSGSSSNVGAGNYVNWFRLIPGSAPKSLIYAATTRASGVPDRFSGSRSGNTATLTISSLQAEDEADYYCSSYDITAVNLFGSGTRLTVLG

SEQ ID NO: 15 - protein, 538 aa, synthetic construct
MALPVTALLLPLALLLHAARPQVRLQGSGPSLVKPSQTLSLTCTVSGFSLTSNAVDWVRQAPGKVPEWLGFIRGGGSTFYNSALKSRLSITRDTSKSQVSLSLSSVTTEDTAVYYCARASCSGDIYTDTCGIDYWGPGLLVTVSSEGKSSGSGSESKVDQSALTQPSSVSRSLGQSVSITCSGSSSNVGAGNYVNWFRLIPGSAPKSLIYAATTRASGVPDRFSGSRSGNTATLTISSLQAEDEADYYCSSYDITAVNLFGSGTRLTVLGTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2024/0316171 A1
(43) Pub. Date: Sep. 26, 2024

(54) COMPOSITIONS AND METHODS FOR TARGETED IMMUNOTHERAPY

(71) Applicant: Dana-Farber Cancer Institute, Inc., Boston, MA (US)

(72) Inventors: Carl Novina, Newton, MA (US); Robert Distel, Framingham, MA (US); Alberto Nobili, Brookline, MA (US); Steven Neier, Waltham, MA (US)

(21) Appl. No.: 18/585,659

(22) Filed: Feb. 23, 2024

Related U.S. Application Data
(63) Continuation of application No. 15/734,116, filed on Dec. 1, 2020, now abandoned.
(60) Provisional application No. 62/680,513, filed on Jun. 4, 2018.

(51) Int. Cl. A61K 39/00; A61K 35/12; A61K 35/17; A61K 38/17; A61K 39/385; A61K 41/00; A61K 45/06; A61K 47/54; A61K 47/68

(52) U.S. Cl. CPC A61K 39/0013; A61K 35/17; A61K 38/1774; A61K 39/385; A61K 41/0042; A61K 41/0057; A61K 45/06; A61K 47/545; A61K 47/555; A61K 47/6889; A61K 47/6897; A61K 2035/124

(57) ABSTRACT

The present invention provides universal immunotherapy compositions useful for targeted treatment of cancers.

### RELATED APPLICATIONS

This application is a Continuation application of U.S. application Ser. No. 15/734,116, filed Dec. 1, 2020, now abandoned, which claims the benefit of priority under 35 U.S.C. § 119(e) to U.S. Provisional Application No. 62/680,513, filed on Jun. 4, 2018, which is incorporated herein by reference in its entirety.

### GOVERNMENT INTEREST

This invention was made with government support under DK105602-01 and T32 AI07386 awarded by the National Institute of Health. The government has certain rights in the invention.

### REFERENCE TO A SEQUENCE LISTING XML

This application contains a Sequence Listing which has been submitted electronically in XML format. The Sequence Listing XML is incorporated herein by reference. Said XML file, created on May 1, 2024, is named 5031461-000149-US2_SL.xml and is 21,494 bytes in size.

### FIELD OF THE INVENTION

The present invention relates generally to universal immunotherapy compositions useful for target treatment of cancers.

### BACKGROUND OF THE INVENTION

Clinical trials have demonstrated that cancer immunotherapies can induce durable responses in patients with advanced cancers. There have been numerous reports of successful treatment of B cell-derived leukemias and lymphomas using chimeric antigen receptor (CAR) T cells. These genetically engineered T cells possess specificity for CD19, a receptor that is prevalent on the surface of cancerous B-cells, but which is also present on normal, non-cancerous B-cells. Thus, CAR-T cells have generated robust immune responses against all cells expressing CD19—including normal B cells.

To date, development of CAR T cell therapies that are effective in treating solid tumors has been thwarted by numerous factors, including the difficulty of identifying true tumor specific antigens (TSAs), namely antigens that are uniquely expressed on tumors but not on untransformed cells. The vast majority of tumor antigens are overexpressed on tumor cells but are also present on normal, non-diseased cells.

### SUMMARY OF THE INVENTION

The present invention circumvents this challenge by leveraging several technological innovations that exploit unique features of a tumor microenvironment. Tumors are known to disrupt the normal cellular microenvironment by over-producing (relative to normal tissue) many molecules that modify the extracellular milieu of the cancer. These include the production of nucleases and proteases, but also, reactive oxygen species (ROS) such as hydrogen peroxide (H₂O₂) and reactive nitrogen species (RNS) such as peroxynitrite (NO₃⁻). The present invention exploits these phenomena efficiently in that it enhances therapeutic efficacy and reduces unintended side effects that cause damage to normal tissue.

The present invention utilizes in its various aspects, bifunctional compounds or complexes that may contain at least two functional domains. One domain, referred to herein as a targeting moiety, binds an antigen on the surface of a tumor cell, also referred to as a tumor associated antigen (TAA). The other domain, referred to herein as a pro-antigen, is designed to be inert in normal (non-diseased) cells and tissues, and to become activated (or “unmasked” or “uncaged”) in the presence of ROS/RNS. The present invention also utilizes chimeric antigen receptor (CAR) T cells which work in concert with the bifunctional compounds. The CAR T cells are equipped with mechanisms that kill cells to which they bind. The CAR T cells of the present invention are designed to be inert to the pro-antigen when the pro-antigen is masked. When the pro-antigen is unmasked (or in activated form), the CAR T cells specifically bind to the pro-antigen. The elevated ROS/RNS levels in the tumor microenvironment, e.g., at the site of the tumor, cause unmasking of the pro-antigen. As a consequence, the CAR T cells selectively target and eliminate tumor cells. Conversely, since normal tissue does not contain elevated levels of ROS/RNS, any pro-antigen that binds the TAA present on normal cells remains in an inactive state which prohibits binding with the CAR-T cells. Thus, normal, non-diseased cells are spared. The efficacy of the present invention may be enhanced, particularly in cases where a tumor does not cause significantly elevated ROS/RNS levels, by administering an ROS/RNS-generating agent to the tumor microenvironment. Enhancement of ROS/RNS in a tumor can occur through local administration of drugs or via systemic delivery. ROS/RNS-enhancing drugs also include drugs that inhibit the breakdown of ROS/RNS. (See, e.g., Xu et al., Cancer Transl. Med. 4(1):35-38 (2018); Yang et al., J. Exp. Clin. Cancer Res. 37:266-275 (2018); Dharmaraja, J. Med. Chem. 60:3221-3240 (2017); de Sá Junior et al. Oxid. Med. Cell Longev. 2017: Article ID 2467940; and Bauer, Anticancer Res. 34:1467-1482 (2014) for central role of elevated ROS in many standard chemotherapy drugs and therapeutic strategies thereof, each of which is incorporated herein by reference.)

Accordingly, a first aspect of the present invention is directed to a bifunctional compound including a pro-antigen covalently linked to a targeting moiety. The pro-antigen, which constitutes one functional modality of the present compounds, is a small molecule having one or more boronic ester protecting groups. The targeting moiety, which constitutes a second functional modality of the present compounds, specifically binds a tumor associated antigen.

Broadly, the pro-antigens of the present invention are small molecules that contain one or more boronic ester protecting groups, and which become deprotected in the presence of ROS or RNS (hereinafter “ROS/RNS). The pro-antigen, which is also referred to herein as a “masked” or “caged” tag, is inert in the sense that it does not bind and thus activate the CAR-T cells unless it becomes deprotected by ROS/RNS.

In some embodiments, the small molecule is a fluorescent molecule such as an anthracene, a fluorescein, an alexa fluor, a rhodamine, a rhodol, an acridine or a xanthene.

In some embodiments, the pro-antigen has a structure represented by formula (A) or (A′):

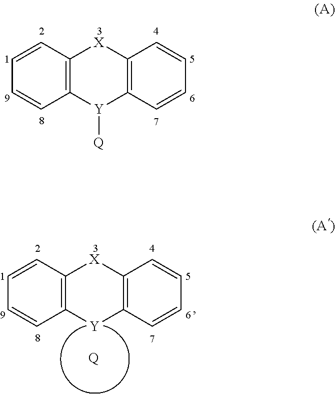

wherein X is C or O, Y is C, the boronic ester protecting group is present at one or more of positions 1-9 and Q represents one or more optionally substituted rings, or a boronic ester protecting group. The optionally substituted rings are 4-7 carbocyclic or heterocyclic rings or a fused ring system, which may be saturated or non-saturated, and wherein heteroatoms may be selected from N, O and S.

In some embodiments, the bifunctional compound has a structure represented by formula (I):

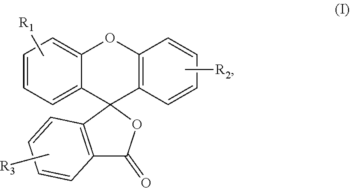

wherein R₁ is O, OH or a boronic ester protecting group;
R₂ is O, OH or a boronic ester protecting group; provided that at least one of R₁ and R₂ is a boronic ester protecting group; and
R₃ is

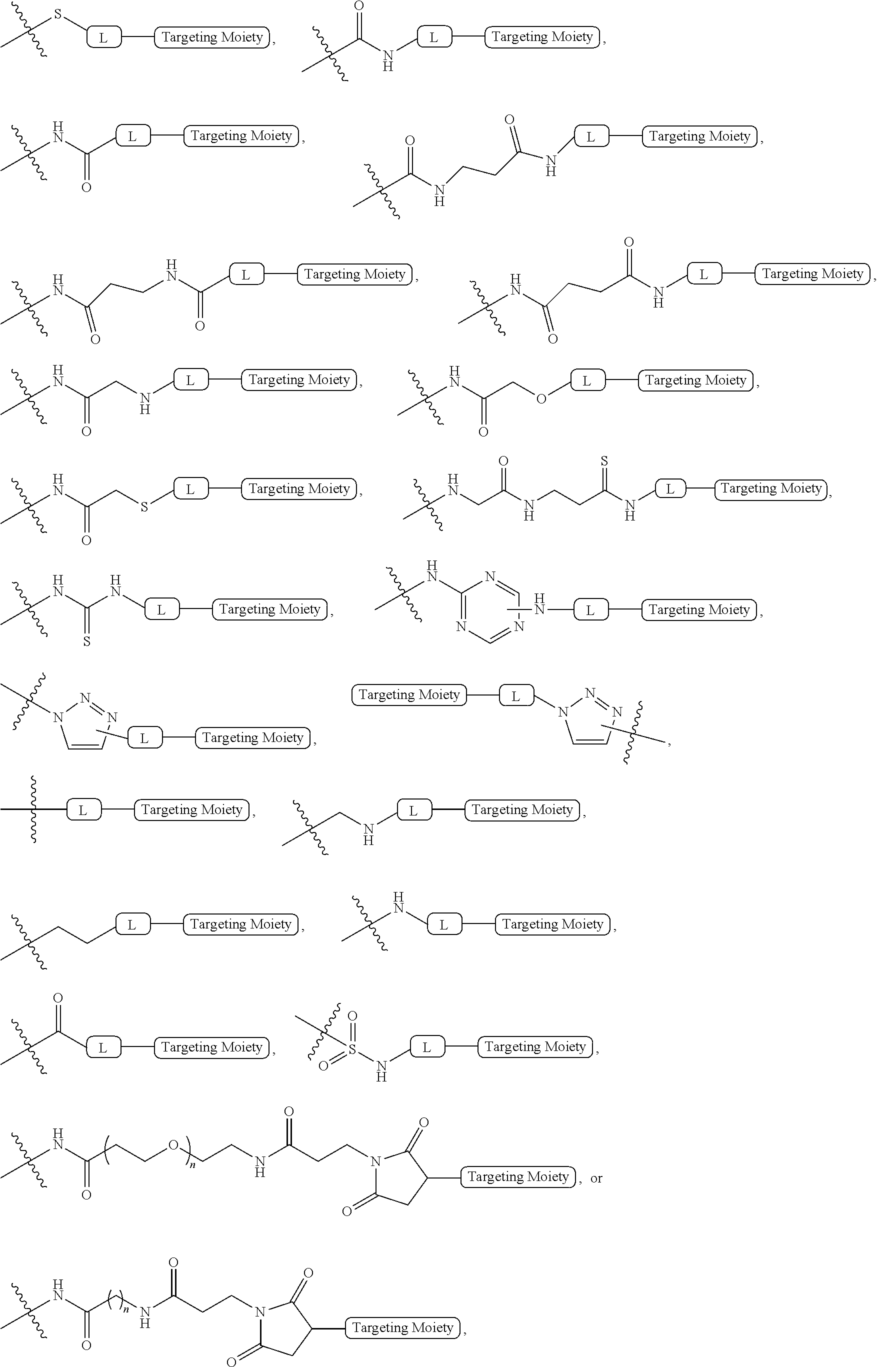

wherein

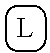

is absent or a linker and n is 1-12: wherein the boronic ester protecting group is

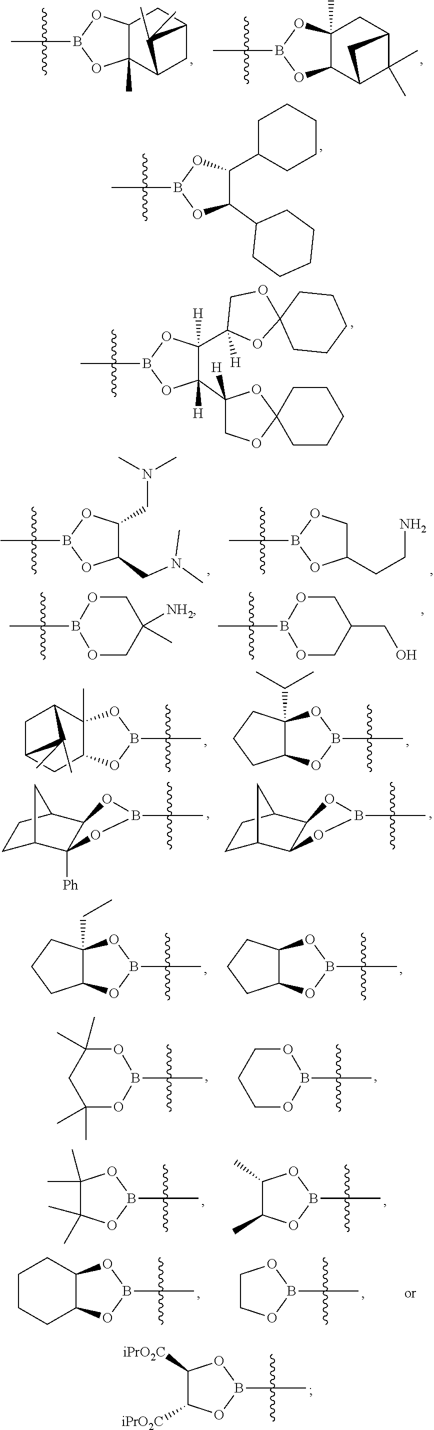

or a stereoisomer thereof.

In some embodiments, each of R₁ and R₂ is a boronic ester protecting group. In some embodiments, the bifunctional compound has a structure represented by formula (Ia):

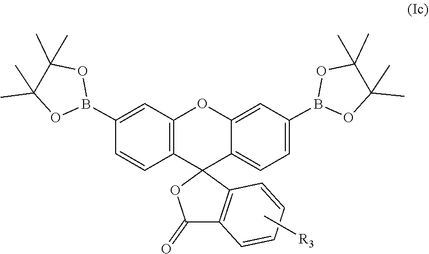

or a stereoisomer thereof.

In some embodiments, the bifunctional compound has a structure represented by formula (Ib):

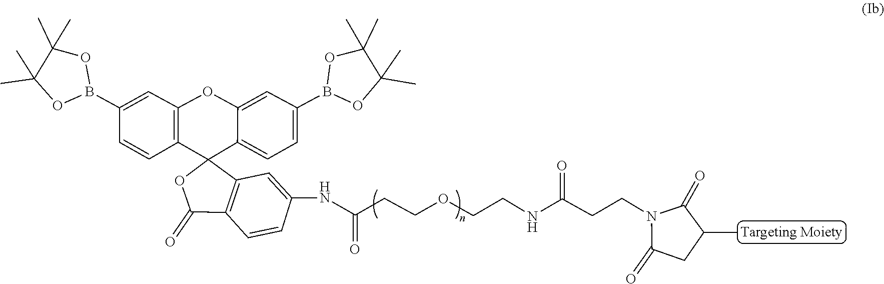

or stereoisomers thereof.

In some embodiments, the bifunctional compound has a structure represented by formula (Ic):

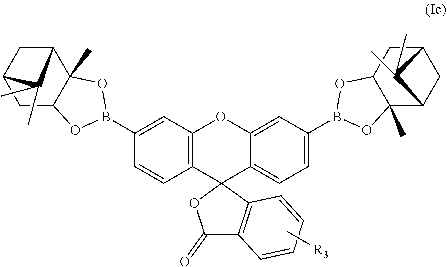

or a stereoisomer thereof.

In some embodiments, the bifunctional compound has a structure represented by formula (Id):

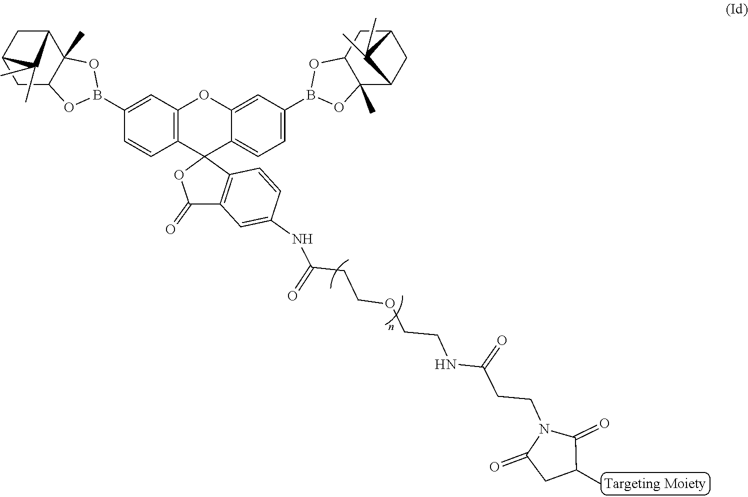

or a stereoisomer thereof.

In some embodiments the bifunctional compound has a structure represented by formula (II):

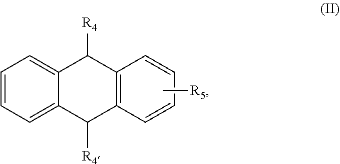

wherein each of R₄ and R_{4′} is independently O or a boronic ester protecting group; provided that at least one of R₄ and R_{4′} is a boronic ester protecting group; and
R₅

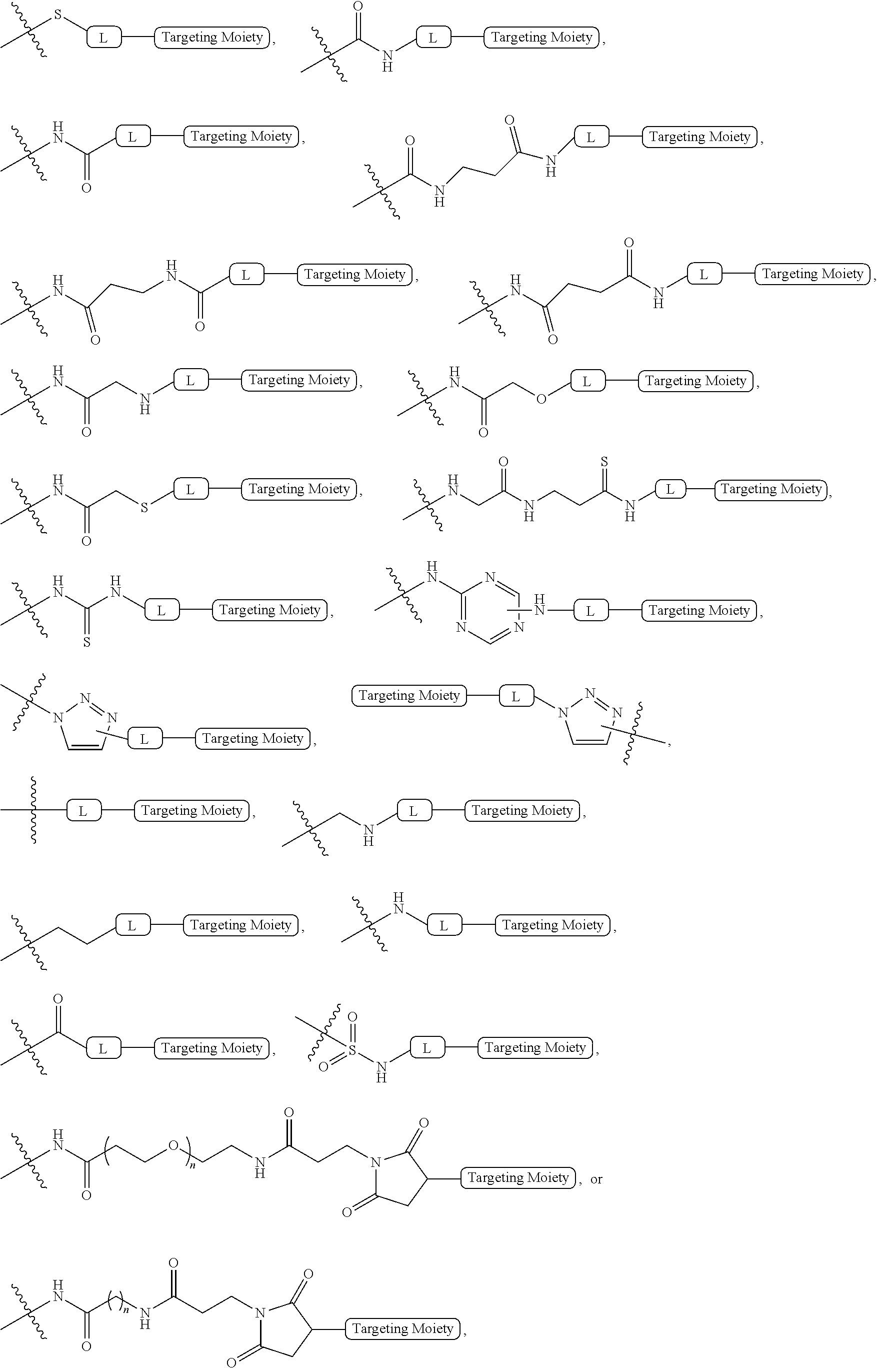

wherein

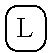

is absent or a linker and n is 1-12: wherein the boronic ester protecting group is

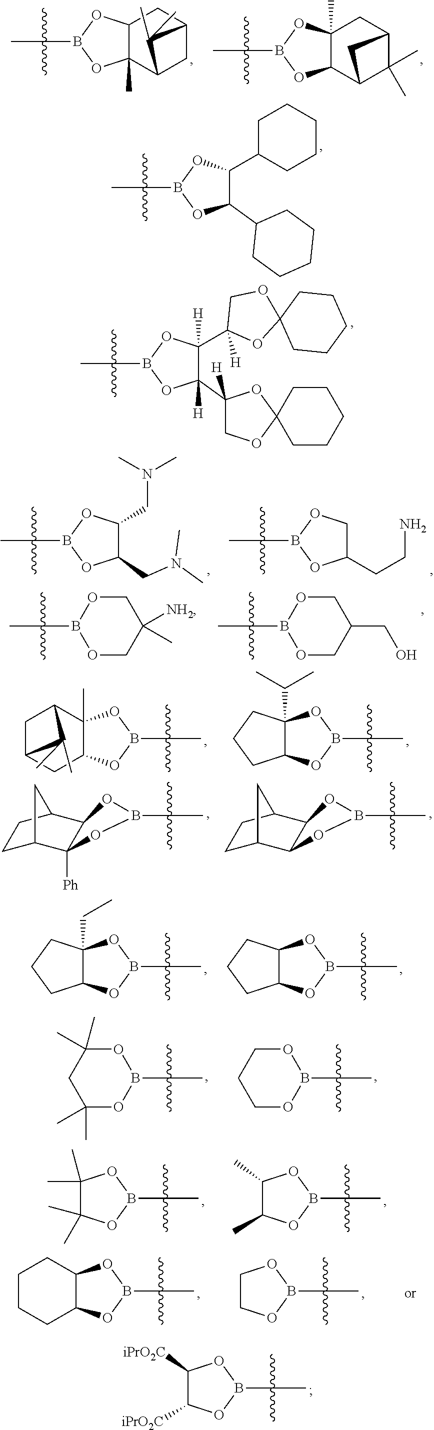

or stereoisomers thereof.

In some embodiments, each of R₄ and R_{4′} is a boronic ester protecting group. In some embodiments, the bifunctional compound has a structure represented by formula (IIa):

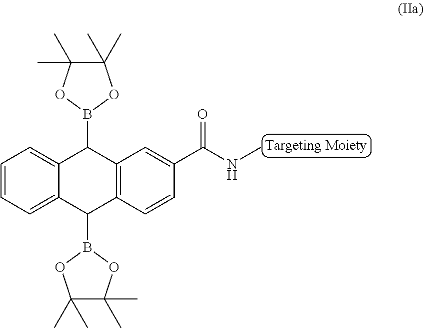 [SMILES: CNC(=O)c1ccc2c(c1)C(B1OC(C)(C)C(C)(C)O1)c1ccccc1C2B1OC(C)(C)C(C)(C)O1]

or a stereoisomer thereof.

In some embodiments, the bifunctional compound has a structure represented by formula (IIb):

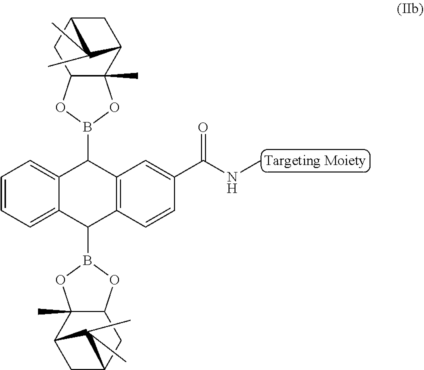 [SMILES: CNC(=O)c1ccc2c(c1)C(B1OC3C[C@@H]4C[C@@H](C4(C)C)[C@]3(C)O1)c1ccccc1C2B1OC2C[C@@H]3C[C@@H](C3(C)C)[C@]2(C)O1]

or a stereoisomer thereof.

In some embodiments, the targeting moiety is an antibody, a functional fragment of an antibody such as a single-chain antibody fragment, a ligand, an aptamer or a nanobody.

In some embodiments, the targeting moiety specifically binds a tumor associated antigen which is platelet derived growth factor receptor alpha (PDGFRα), activin a receptor type 1 (ACVR1), human epidermal growth factor receptor 2 (HER2), prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes: MART-1), myo-D1, muscle-specific actin (MSA), neurofilament, neuron-specific enolase (NSE), placental alkaline phosphatase, synaptophysis, thyroglobulin, thyroid transcription factor-1, dimeric form of pyruvate kinase isoenzyme type M2 (tumor M2-PK), an abnormal ras protein, an abnormal p53 protein, EGFRvIII, diganglioside GD2, mesothelin, interleukin 13 receptor α 2 (IL13Rα2), fibroblast activation protein (FAP), CD133, natural-killer group 2, member D (NKG2D), Ephrin type-A receptor 2 (EphA2), CD70, chondroitin sulfate proteoglycan 4 (CSPG4), CD56, CS-1, CD38, CD138, B-cell maturation antigen (BCMA) or L1 cell adhesion molecule (L1CAM).

In another aspect, pharmaceutical compositions of a therapeutically effective amount of the bifunctional compound or a stereoisomer thereof, and a pharmaceutically acceptable carrier are also provided.

In another aspect, methods of making the bifunctional are also provided.

In another aspect, kits containing a therapeutically effective amount of one or more of the bifunctional compounds are provided. In some embodiments, the kits also contain reagents for producing autologous CAR-T cells that specifically recognize an unmasked compound of the invention. In some embodiments, the kits contain a therapeutically effective amount of one or more of the bifunctional compounds and a therapeutically effective amount (number) of allogeneic CAR-T cells that specifically bind the pro-antigen in deprotected (unmasked) form. In some embodiments, the kits further include one or more ROS/RNS-generating agents.

In further aspects, methods of treating cancer are provided, which include administering to a subject in need thereof, a therapeutically effective amount of a bifunctional compound and a therapeutically effective number of CAR-T cells, wherein the CAR-T cells include an extracellular ligand that specifically binds the pro-antigen in deprotected (unmasked) form. i.e. the CAR-T cells specifically bind the tag.

In some embodiments, the methods treat solid tumors. They may further entail locally administering to the subject, to the tumor microenvironment, a reactive oxygen species (ROS) or reactive nitrogen species (RNS)-generating agent, thereby deprotecting (unmasking) the fused ring system. For example, a ROS-generating agent results in increased quantities of hydrogen peroxide, superoxide, hydroxyl radical or hypochlorous acid in a tumor microenvironment. For example, a RNS-generating agent results in increased quantities of peroxynitrite in a tumor microenvironment.

In some embodiments, the agent includes ultrasound, electromagnetic stimulation, a reactive chemical species-enhancing drug, radionuclide, external beam radiation, brachytherapy, lanthanide metal nanoparticles or a combination of two or more thereof. In some embodiments, the agent is a CD44 inhibitor.

In some embodiments, the compound is administered to the subject prior to administration of the CAR-T cells. In other embodiments, the compound is administered to the subject after administration of the CAR-T cells. In still other embodiments, the compound is administered to the subject concomitantly with administration of the CAR-T cells.

In some embodiments, the bifunctional compound is administered at a dose of 0.01 mg/kg to 500 mg/kg body weight. In some embodiments, the bifunctional compound is administered parenterally. In some embodiments, the CAR-T cells are administered at a dose of 10⁴ to 10⁹ cells per kg body weight. In some embodiments, the CAR-T cells are administered parenterally. In some embodiments, the compound is administered more than once, e.g., at least two times, at least three times, at least four times, at least five times, at least six times, at least seven times, at least eight times, at least nine times, or at least ten times, and the CAR-T cells are administered once. In some embodiments, the bifunctional compound and CAR-T cells are administered more than once, e.g., at least two times, at least three times, at least four times, at least five times, at least six times, at least seven times, at least eight times, at least nine times, or at least ten times. In some embodiments, the bifunctional compound, the ROS/RNS-generating agent and CAR-T cells are administered more than once, e.g., at least two times, at least three times, at least four times, at least five times, at least six times, at least seven times, at least eight times, at least nine times, or at least ten times. In some cases, subsequent administration of either the compound, the agent, and/or the CAR-T cells is via a different route of administration as compared to the first administration of the compound, the agent, and/or the CAR-T cells, respectively. In some embodiments, the bifunctional compound and CAR-T cells are administered through various parenteral routes of administration, e.g., intravenously, intratumorally, intradermally, subcutaneously (s.c., s.q., sub-Q, Hypo), intramuscularly (i.m.), intraperitoneally (i.p.), intra-arterially, intramedullarilly, intracardially, intra-articularly, intrasynovially, intracranially, intraspinally, and intrathecally. In some embodiments, the bifunctional compound, the ROS/RNS-generating agent and CAR-T cells are administered through various parenteral routes of administration, e.g., intravenously, intratumorally, intradermally, subcutaneously (s.c., s.q., sub-Q, Hypo), intramuscularly (i.m.), intraperitoneally (i.p.), intra-arterially, intramedullarilly, intracardially, intra-articularly, intrasynovially, intracranially, intraspinally, and intrathecally.

### BRIEF DESCRIPTION OF THE DRAWINGS

FIG. 1 is a chemical schematic flow diagram of boronated fluorescein being unmasked/uncaged upon exposure to a reactive oxygen species.

FIG. 2 is a representation of a chemical model showing binding of 4M5.3 antibody to fluorescein (FL).

FIG. 3A is a chemical schematic flow diagram of the synthesis of a pro-antigen of the invention. The steps include: 1.1) adding reactant K₂CO₃ in solvent DMF for 5 minutes at 0° C.; and 1.2) 17 hours at room temperature.

FIG. 3B is a chemical schematic flow diagram of the synthesis of a pro-antigen of the invention. The steps include: 1.1) adding reactant K₂CO₃ in solvent DMF for 15 minutes at (° C.); and 1.2) 1 hour at 0° C.); 4 hours at room temperature.

FIG. 4A is a chemical schematic flow diagram of the synthesis of a pro-antigen of the invention. The steps include: 1.1) adding reactant EtN(Pr-i)₂ in solvent DMF for 72 hours at room temperature in the dark; and 2.1) performing Miyaura borylation reaction with reactant AcOK, catalyst 95464-05-4 in solvents dioxane and CH₂Cl₂, 6 hours reflux; overnight at room temperature for an overall yield of 42%.

FIG. 4B is a chemical schematic flow diagram of the synthesis of a pro-antigen of the invention. The steps include: 1.1) adding reactant EtN(Pr-i)₂ in solvent DMF for 72 hours at room temperature in the dark; and 2.1) performing Miyaura borylation reaction with reactant AcOK, catalyst [1,1′-bis(diphenylphosphino)ferrocene]dichloropalladium(II) dichloromethane adduct (CAS #95464-05-4) in solvents dioxane and CH₂Cl₂, 6 hours reflux; overnight at room temperature.

FIG. 5A is a chemical schematic flow diagram of the synthesis of a pro-antigen of the invention. The steps include: 1.1) adding reactant 1-ethyl-3-(3-dimethylaminopropyl)carbodiimide hydrochloride (EDC) in solvent DMF for 48 hours at room temperature: 2.1) adding additional DMF for 24 hours at room temperature: 3.1) adding reactant AcOK and catalyst [1,1′-bis(diphenylphosphino)ferrocene]dichloropalladium(II) (CAS #72287-26-4) in solvent dioxane for 2 hours at 50° C.: 4.1) adding reactant F₃CCO₂H in solvent MeOH overnight at room temperature; and 4.2) adding reactant Et₃N in solvent MeOH overnight at room temperature.

FIG. 5B is a chemical schematic flow diagram of the synthesis of a pro-antigen of the invention. The steps include: 1.1) adding additional DMF for 24 hours at room temperature: 2.1) adding reactant AcOK and catalyst [1,1′-bis (diphenylphosphino)ferrocene]dichloropalladium(II) in solvent dioxane for 2 hours at 50° C.: 3.1) adding reactant F₃CCO₂H in solvent MeOH overnight at room temperature; and 3.2) adding reactant Et₃N in solvent MeOH overnight at room temperature.

FIG. 5C is a chemical schematic flow diagram of the synthesis of a pro-antigen of the invention. The steps include: 1.1) adding reactant EDC in solvent DMF for 48 hours at room temperature: 2.1) adding additional DMF for 24 hours at room temperature; and 3.1) adding reactant AcOK and catalyst [1,1′-bis(diphenylphosphino)ferrocene]-dichloropalladium(II) in solvent dioxane for 2 hours at 50° C.

FIG. 5D is a chemical schematic flow diagram of the synthesis of a pro-antigen of the invention. The steps include: 1.1) adding reactant AcOK and catalyst [1,1′-bis(diphenylphosphino)ferrocene]dichloropalladium(II) in solvent dioxane for 2 hours at 50° C.: 2.1) adding reactant F₃CCO₂H in solvent MeOH overnight at room temperature; and 2.2) adding reactant Et₃N in solvent MeOH overnight at room temperature.

FIG. 5E is a chemical schematic flow diagram of the synthesis of a pro-antigen of the invention. The steps include: 1.1) adding additional DMF for 24 hours at room temperature; and 2.1) adding reactant AcOK and catalyst [1,1′-bis(diphenylphosphino)ferrocene]dichloropalladium(II) in solvent dioxane for 2 hours at 50° C.

FIG. 5F is a chemical schematic flow diagram of the synthesis of a pro-antigen of the invention. The steps include: 1.1) adding reactant EDC in solvent DMF for 48 hours at room temperature; and 2.1) adding additional DMF for 24 hours at room temperature.

FIG. 5G is a chemical schematic flow diagram of the synthesis of a pro-antigen of the invention. The steps include: 1.1) adding reactant F₃CCO₂H in solvent MeOH overnight at room temperature; and 1.2) adding reactant Et₃N in solvent MeOH overnight at room temperature.

FIG. 5H is a chemical schematic flow diagram of the synthesis of a pro-antigen of the invention, and includes: 1.1) adding reactant AcOK and catalyst [1,1′-bis(diphenylphosphino)ferrocene]dichloropalladium(II) in solvent dioxane for 2 hours at 50° C.

FIG. 5I is a chemical schematic flow diagram of the synthesis of a pro-antigen of the invention, and includes: 1.1) adding DMF for 24 hours at room temperature.

FIG. 5J is a chemical schematic flow diagram of the synthesis of a pro-antigen of the invention, and includes: adding reactant EDC in solvent DMF for 48 hours at room temperature.

FIG. 6 is a drawing showing that anti-FL CAR-T cells will not recognize a caged FL molecule attached to an antibody that recognizes a tumor associated antigen.

FIG. 7 is a drawing depicting the unmasking of FL conjugated to a patient tumor targeting antibody by elevated levels of ROS/RNS in the microenvironment of a tumor and/or by administration of a ROS/RNS-generating agent to cleave the protecting group.

FIG. 8 is a drawing depicting recognition of FL by anti-FL CAR-T cells after unmasking by exposure to elevated levels of ROS/RNS in the microenvironment of a tumor and/or by administration of a ROS/RNS-generating agent to cleave the protecting group.

FIG. 9A is an image of the ultraviolet/visible (UV/Vis) spectrum of 40 μM compound 1 in 50 mM 4-(2-hydroxyethyl)-1-piperazineethanesulfonic acid (HEPES) pH 7, 2% DMSO. Absorbance at 280 nM was linear up to at least 40 μM with and extinction coefficient of 2230 M⁻¹ cm⁻¹.

FIG. 9B is an image of the UV/Vis spectrum of 60 μM compound 2 in 50 mM HEPES pH 7. Absorbance at 495 was linear with concentration up to at least 60 μM with an extinction coefficient of 3980 M⁻¹ cm⁻¹.

FIG. 9C is a fluorescence emission spectrum of 20 μM compound 1 and 15 μM compound 2 in 50 mM HEPES, pH 7. The excitation wavelength was 450 nm.

FIG. 9D is a fluorescence excitation spectrum of compound 2 in 50 mM HEPES, pH 7. Emission was monitored at 520 nm.

FIG. 10 is a graph of a time-course of the kinetic response of compound 1 to H₂O₂ at 100 mM H₂O₂. Solid line is a two-phase association fit to the data.

FIG. 11A is a chemical schematic flow diagram of the generation of uncaged compound (2) from compound 1 after the exposure to radiation.

FIG. 11B is a graph showing the extent of compound 1 uncaging in relation to the amount of radiation provided and the presence of cells in solution.

FIG. 12A is a chemical schematic flow diagram of the generation of an uncaged molecule (4) from compound 3 after the exposure to ROS.

FIG. 12B is a graph showing the extent of molecule uncaging in relation to the amount of ROS (in this case exogenous H₂O₂) available in solution. A 20 μM solution of compound 3 was mixed with H₂O₂ at concentrations between 50 and 1000 μM. The production of compound 4 was monitored by fluorescence-detected HPLC after 15 minutes reaction.

### DETAILED DESCRIPTION OF THE INVENTION

Unless defined otherwise, all technical and scientific terms used herein have the same meaning as is commonly understood by one of skill in art to which the subject matter herein belongs. As used in the specification and the appended claims, unless specified to the contrary, the following terms have the meaning indicated in order to facilitate the understanding of the present invention.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below: In the case of conflict, the present specification, including definitions, will control. In addition, the materials, methods, and examples are illustrative only and not intended to be limiting.

As used herein, the term “cyclic” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl. 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula-R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O. N. N(O), S. S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and Spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl. 6-azabicyclo[3.1.1]heptanyl. 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl: 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl: pyrimidyl, such as pyrimid-2-yl and pyrimid-4-yl: triavinyl, such as 1,3,4-triazin-2-yl and 1,3,5-triazin-4-yl: pyridazinyl, in particular pyridazin-3-yl, and pyrazinyl. The pyridine N-oxides and pyridazine

N-oxides and the pyridyl, pyrimid-2-yl, pyrimid-4-yl, pyridavinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl. 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperavinyl, and 2- or 3-pyrrolidinyl. The term heterocyclic also embraces heterocyclylalkyl groups which as disclosed above refer to a group of the formula-R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula-R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—Re-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 4-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridavinyl, triavinyl, tetravinyl, tetrazolo[1,5-b]pyridavinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methy lenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenavinyl, phenothiazinyl, phenoxavinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula-R^{c}-heteroaryl, wherein R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc. Representative substituents include halogens, hydroxyl groups, and any other organic groupings containing any number of carbon atoms, e.g., 1-14 carbon atoms, and which may include one or more (e.g., 1 2 3, or 4) heteroatoms such as oxygen, sulfur, and nitrogen grouped in a linear, branched, or cyclic structural format.

Representative examples of substituents may thus include alkyl, substituted alkyl, alkoxy, substituted alkoxy, alkenyl, substituted alkenyl, alkynyl, substituted alkynyl, cyclic, substituted cyclic, carbocyclic, substituted carbocyclic, heterocyclic, substituted heterocyclic, aryl (e.g., benzyl and phenyl), substituted aryl (e.g., substituted benzyl or phenyl), heteroaryl, substituted heteroaryl, aralkyl, substituted aralkyl, halo, hydroxyl, aryloxy, substituted aryloxy, alkylthio, substituted alkylthio, arylthio, substituted arylthio, cyano, carbonyl, substituted carbonyl, carboxyl, substituted carboxyl, amino, substituted amino, amido, substituted amido, sulfonyl, substituted sulfonyl, amino acid, and peptide groups.

The term “tumor-associated antigen” (TAA), which may also be referred to as a “tumor antigen” or a “cancer associated antigen”, refers to a molecule (typically a protein, carbohydrate or lipid) that is expressed on the surface of a cancer cell, either entirely or as a fragment (e.g., major histocompatibility complex (MHC)/peptide), and which may be preferentially targeted by a pharmacological agent to the cancer cell.

As used herein, the term “affimer” refers to a small and highly stable protein engineered to display peptide loops which provide a high affinity binding surface for a specific target protein. It is a protein of low molecular weight, 12-14 kDa, derived from the cysteine protease inhibitor family of cystatins.

The term “aptamer” refers to an oligonucleotide (e.g., DNA, RNA, or an analog or derivative thereof) that binds to a particular target, such as a polypeptide.

The term “bind” or “binding.” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Biological binding” defines a type of interaction that occurs between pairs of molecules including proteins, nucleic acids, glycoproteins, carbohydrates, hormones, or the like. The term “binding partner” refers to a molecule that can undergo binding with a particular molecule. “Specific binding” refers to molecules that are able to bind to or recognize a binding partner (or a limited number of binding partners) to a substantially higher degree than to other, similar biological entities.

The term “effector function” refers to a specialized function of a cell, which in the case of T cells, includes induction of cytokine and chemokine production, as well as activation of the cytolytic activity of the cells.

The present invention relates to universal immunotherapy systems/kits, compositions and methods of treating cancer.

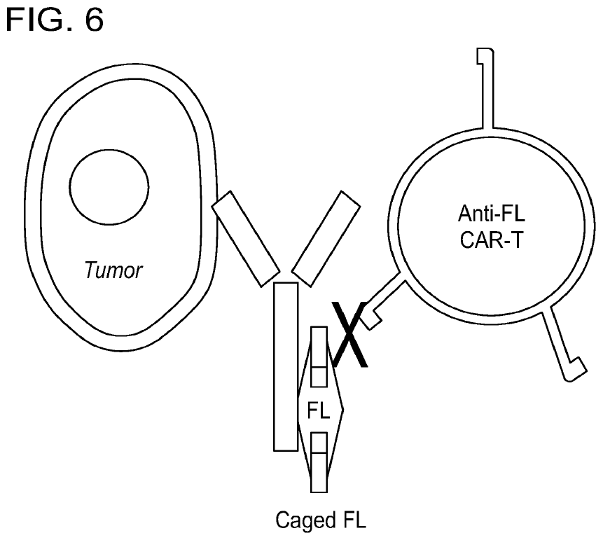

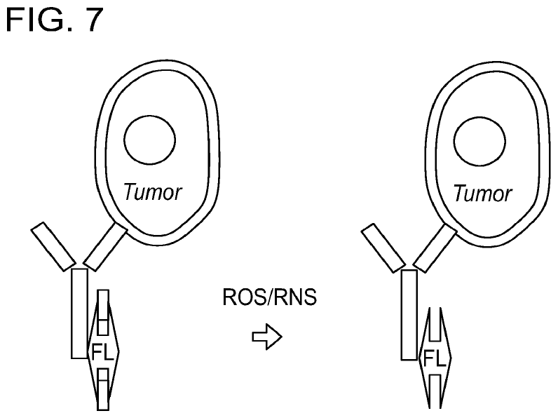

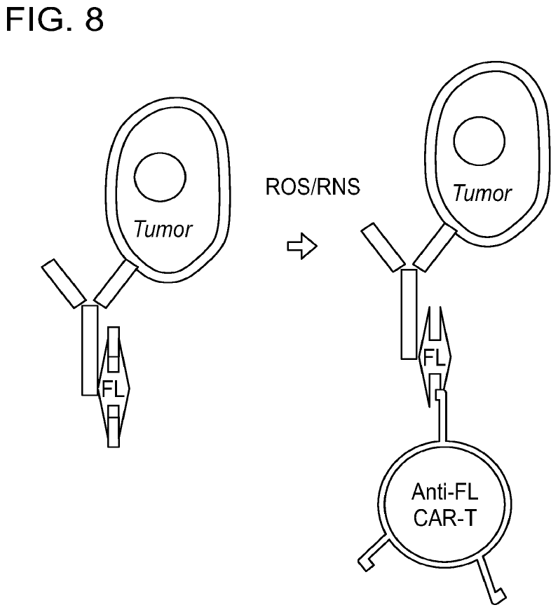

As schematically illustrated in FIG. 6-FIG. 8, two events determine whether CAR T cells will be activated by the synthetic small molecule. These Binary Activated T cells using Chimeric Antigen Receptors (BAT-CARs) are ineffective in the presence of the masked small molecule. They become activated only at sites where the small molecule is unmasked. The systems and compositions of the invention can be tailored to direct T cell responses against any solid tumor in a patient-specific fashion.

Another aspect of this invention is that any chimeric cellular receptor can be engineered to be stimulated by administration of a small molecule. That is, fusion of a single-chain antibody with any cellular receptor, can produce novel chimeric receptors. Thus, administration of a small molecule recognized by the single chain antibody can stimulate downstream effects in the target cell characteristic of stimulating the receptor with its natural ligand. In the current invention, T cell receptor signaling can be enabled by administration of a small molecule bound to a tumor targeting ligand. That is, administration of a small molecule recognized by the single-chain antibody fused to T cell signaling molecules (for example but not uniquely CD28 and CD3ζ) leads to hallmark changes in T cells representative to T cell receptor signaling. By making chimeras of a small molecule binding single chain antibody with any cellular receptor, specific biological outcomes can be induced by administration of a small molecule recognized by the single chain antibody.

### Bifunctional Compounds

### Targeting Moiety

The targeting moiety, which constitutes one of the functional modalities of the bifunctional compounds of the present invention, specifically binds a tumor associated antigen.

Targeting moieties according to the invention have binding specificity for tumor associated antigens that may be present on a cancer cell. In some embodiments, the TAA is a marker expressed by both normal cells and cancer cells, e.g., a lineage marker, e.g., CD19 on B cells. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, the tumor associated antigen is a major histocompatibility complex (MHC) presented peptide. Normally, peptides derived from endogenous proteins fill the pockets of MHC class I molecules, and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell.

Representative examples of targeting moieties include antibody molecules and functional (i.e., antigen-binding) fragments thereof, receptor ligands, peptides, haptens, aptamers, affimers, T-cell receptor tetramers and other targeting molecules known to those skilled in the art. For example, the targeting moiety may include a nucleic acid, polypeptide, glycoprotein, carbohydrate, or lipid.

In certain embodiments, the targeting moiety is an antibody or antibody fragment. Representative examples of antibodies include monoclonal antibodies, polyclonal antibodies, Fv, Fab, Fab′ and F(ab′)₂ immunoglobulin fragments, synthetic stabilized Fv fragments, e.g., single chain Fv fragments (scFv), disulfide stabilized Fv fragments (dsFv), single variable region domains (dAbs) minibodies, combibodies, and multivalent antibodies such as diabodies and multi-scFv, single domains from camelids or engineered human equivalents. The term “antibody” also includes any protein having a binding domain which is homologous or largely homologous to an immunoglobulin binding domain. Such proteins may be derived from natural sources, or partly or wholly synthetically produced.

In certain embodiments, the targeting moiety is an affimer. Affimer proteins are composed of a scaffold, which is a stable protein based on the cystatin protein fold. They display two peptide loops and an N-terminal sequence that can be randomized to bind different target proteins with high affinity and specificity similar to antibodies. Stabilization of the peptide upon the protein scaffold constrains the possible conformations which the peptide may take, thus increasing the binding affinity and specificity compared to libraries of free peptides.

In certain embodiments, a targeting moiety is a nucleic acid molecule (e.g. an aptamer) that binds to a cell type specific marker. Aptamers are short synthetic single-stranded oligonucleotides that specifically bind to various molecular targets such as small molecules, peptides, proteins, nucleic acids, and even cells and tissues. These small nucleic acid molecules can form secondary and tertiary structures capable of specifically binding proteins or other cellular targets, and are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment) (Ellington et al. Nature 346(6287):818-822 (1990); Tuerk et al., Science 249(4968):505-510 (1990); Ni et al., Curr. Med. Chem. (2011); 18(27):4206-14. Methods of generating an aptamer for any given target are well known in the art.

In some embodiments, a targeting moiety is a naturally occurring or synthetic ligand for a cell surface receptor.

In some embodiments, the targeting moiety is a carbohydrate. Carbohydrates may be natural or synthetic. A carbohydrate may be a derivatized natural carbohydrate. In some embodiments, the carbohydrate comprises monosaccharide or disaccharide, including but not limited to, glucose, fructose, galactose, ribose, lactose, sucrose, maltose, trehalose, cellbiose, mannose, xylose, arabinose, glucoronic acid, galactoronic acid, mannuronic acid, glucosamine, galatosamine, or neuramic acid. In some embodiments, the carbohydrate is a polysaccharide, such as, but not limited to, pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, konjac, glucommannan, pustulan, heparin, hyaluronic acid, curdlan, and xanthan. In some embodiments, the carbohydrate is a sugar alcohol, such as, but not limited to mannitol, sorbitol, xylitol, erythritol, maltitol, or lactitol.

In certain embodiments, the targeting moiety is directed to a TAA expressed by tumors that generate elevated levels of ROS and/or RNS. Elevated production of reactive chemical species (ROS/RNS) have been detected in almost all cancers, where they promote many aspects of tumor development and progression (Liou et al., Free Radic. Res. 44: 479-496 (2010); Trachootham et al., Nat. Rev. Drug Discov. 8: 579-591 (2009)). Altered cellular metabolism is considered a hallmark of cancer and is fast becoming an avenue for therapeutic intervention. Mitochondria have recently been viewed as an important cellular compartment that fuels the metabolic demands of cancer cells, as mitochondria are the major source of ATP and metabolites necessary to fulfill the bioenergetics and biosynthetic demands of cancer cells. Furthermore, mitochondria are central to cell death and the main source for generation of reactive oxygen species (ROS; Chowdhury et al., Oxid. Med. Cell. Longer. 2018: 1-10 (2018); Zhang et al., Oxid. Med. Cell. Longev. 2016: 1616781 (2016)). An extensive analysis of tumor cell lines in vitro has shown that they are regularly characterized by i) extracellular superoxide anion generation and ii) expression of membrane-associated catalase that protects the cells against intercellular ROS signaling and apoptosis.

Representative examples of targeting moieties and their corresponding receptors on tumor cells are set forth in Table 1:

TABLE 1

| TAA | Targeting Moiety | Source |
|---|---|---|
| CD99 | mAb 3B2/TA8, MEM- | Invitrogen ™; Bio-Rad |
| | 131; mAb DN16 | |
| PDGFRα | mAb APAS; mAb | Invitrogen ™; Biolegend |
| | 16A1 | |
| ACVR1 | mAb C-5 | Santa Cruz Biotechnology |
| CD22 | Antibody or fragment | Haso et al., Blood, 121(7): 1165-1174 |
| | thereof | (2013); Wayne et al., Clin. Cancer Res. |
| | | 16(6): 1894-1903 (2010); Kato et al., |
| | | Leuk Res 37(1): 83-88 (2013); Creative |
| | | BioMart (creativebiomart.net): MOM- |
| | | 18047-S(P) |
| CD33 | Gemtuzumab | Bross et al, Clin. Cancer Res. 7(6): 1490- |
| | Ozogamicin, or hP67.6 | 1496 (2001) |
| CD33 | Lintuzumab of | Caron et al., Cancer Res. 52(24): 6761- |
| | HuM195 | 6767 (1992) |
| CD33 | AVE9633 | Lapusan et al., Invest New Drugs |
| | | 30(3): 1121-1131 (2012) |
| GD2 | Antibody or fragment | Mujoo et al., Cancer Res. 47(4): 1098- |
| | thereof | 1104 (1987); Cheung et al., Cancer Res |
| | | 45(6): 2642-2649 (1985), Cheung et al., J. |
| | | Clin. Oncol. 5(9): 1430-1440 (1987), |
| | | Cheung et al., J. Clin. Oncol. 16(9): 3053- |
| | | 3060 (1998), Handgretinger et al., Cancer |
| | | Inmunol. Immunother. 35(3): 199-204 |
| | | (1992); US Publication No.: 20100150910 |
| | | of PCT Publication No.: WO 2011160119 |
| GD2 | mAb 14.18, 14G2a, | WO2012033885, WO2013040371, |
| | ch14.18, hul4.18, 3FS, | WO2013192294, WO2013061273, |
| | hu3F8, 3G6, 8B6, | WO2013123061, WO2013074916, and |
| | 60C3, 10B8, ME36.1, | WO201385552 |
| Tn antigen | Antibody or fragment | U.S. Pat. No. 8,440,798, Brooks et al., |
| | thereof | PNAS 107(22): 10056-10061 (2010), |
| | | and Stone et al., OncoImmunology |
| | | 1(6): 863-873(2012) |
| PSMA | Antibody or fragment | Parker et al., Protein Expr. Purif. |
| | thereof | 89(2): 136-145 (2013) |
| PSMA | J591 ScFv | US 20110268656 |
| PSMA | scFvD2B | Frigerio et al., European J. Cancer |
| | | 49(9): 2223-2232 (2013) |
| PSMA | mAbs 3/A12, 3/E7 and | WO 2006125481 |
| | 3/F11 and single chain | |
| | antibody fragments | |
| | scFv A5 and D7 | |
| ROR1 | Antibody or fragment | Hudecek et al., Clin. Cancer Res. |
| | thereof | 19(12): 3153-3164 (2013); WO |
| | | 2011159847; and US20130101607 |
| TAG72 | Antibody or fragment | Hombach et al., Gastroenterology |
| | thereof | 113(4): 1163-1170 (1997) |
| TAG72 | Ab691 | Abcam ® |
| FAP | Antibody or fragment | Ostermann et al., Clinical Cancer |
| | | Research 14: 4584-4592 (2008) |
| FAP | sibrotuzumab | Hofheinz et al., Oncology Research |
| | | and Treatment 26(1), 2003); and Tran |
| | | et al., J. Exp. Med. 210(6): 1125-1135 |
| | | (2013) |
| CD44v6 | Antibody or fragment | Casucci et al., Blood 122(20): 3461- |
| | therof | 3472 (2013) |
| CEA | Antibody or fragment | Chmielewski et al., Gastoenterology |
| | thereof | 143(4): 1095-1107 (2012) |
| EPCAM | MT110, EpCAM-CD3 | clinicaltrials.gov/ct2/show/NCT00635596) |
| | bispecific Ab | |
| EPCAM | Edrecolomab; | Commercial sources |
| | 3622W94; ING-1; and | |
| | adecatumumab | |
| | (MT201) | |
| PRSS21 | Antibody or fragment | U.S. Pat. No. 8,080,650 |
| | therof | |
| B7H3 (also known as | MGA271 | Macrogenics |
| CD276) | | |
| KIT (also known as | Antibody or fragment | U.S. Pat. No. 7,915,391, |
| CD117) | thereof | US20120288506, and several |
| | | commercial catalog antibodies |
| IL-13Ra2 | Antibody or fragment | WO2008/146911, WO2004087758, |
| | thereof | several commercial catalog antibodies, |
| | | and WO2004087758 |
| CD30 | Antibody or fragment | U.S. Pat. No. 7,090,843 B1, and |
| | thereof | EP0805871 |
| GD3 | Antibody or fragment | U.S. Pat. No. 7,253,263; U.S. Pat. No. |
| | thereof | 8,207,308; US 20120276046; |
| | | EP1013761; WO2005035577; and |
| | | U.S. Pat. No. 6,437,098 |
| CD171 | Antibody or fragment | Hong et al., J. Immunother. 37(2): 93- |
| | thereof | 104 (2014) |
| IL-11Ra | Antibody or fragment | Abcam ® (cat# ab55262) or Novus |
| | thereof | Biologicals (cat# EPR5446) |
| IL-11Ra | Peptide | Huang et al., Cancer Res. 72(1): 271- |
| | | 281 (2012) |
| PSCA | scFv 7F5 | Morgenroth et al., Prostate |
| | | 67(10): 1121-1131 (2007) |
| PSCA | ScFv CS-II | Nejatollahi et al., J. of Oncology |
| | | 2013(2013), article ID 839831 |
| PSCA | Antibody or fragment | US Pat Publication No. 20090311181 |
| | thereof | |
| VEGFR2 | Antibody or fragment | Chinnasamy et al., J. Clin. Invest. |
| | therof | 120(11): 3953-3968 (2010) |
| LewisY | Hu3S193 Ab (scFvs) | Kelly et al., Cancer Biother |
| | | Radiopharm. 23(4): 411-423 (2008) |
| LewisY | NC10 scFy | Dolezal et al., Protein Engineering |
| | | 16(1): 47-56 (2003) |
| CD24 | Antibody or fragment | Maliar et al., Gastroenterology |
| | thereof | 143(5): 1375-1384 (2012) |
| PDGFR-beta | ab32570 | Abcam ® |
| SSEA-4 | Antibody MC813 | Cell Signaling; or other commercially |
| | | available antibodies |
| Folate receptor alpha | Antibody IMGN853 | Commercial sources |
| Folate receptor alpha | Antibody or fragment | US20120009181; U.S. Pat. No. |
| | thereof | 4,851,332, LK26: U.S. Pat. No. |
| | | 5,952,484 |
| ERBB2 (Her2/neu) | trastuzumab, or | Commercial sources |
| | pertuzumab | |
| MUC1 | Antibody SAR566658 | Commercial sources |
| EGFR | cetuximab, | Commercial sources |
| | panitumumab, | |
| | zalutumumab, | |
| | nimotuzumab, or | |
| | matuzumab | |
| NCAM | antibody clone 2-2B: | EMD Millipore |
| | MAB5324 | |
| Ephrin B2 | Antibody or fragment | Abengozar et al., Blood 119(19): 4565- |
| | thereof | 4576 (2012) |
| IGF-1 | Antibody or fragment | U.S. Pat. No. 8,344,112 B2; |
| | thereof | EP2322550 A1; WO 2006/138315, or |
| | | PCT/US2006/022995 |
| CAIX | antibody clone 303123 | R&D Systems |
| LMP2 | Antibody or fragment | U.S. Pat. No. 7,410,640, or |
| | thereof | US20050129701 |
| Gp100 | antibody HMB45, | Commercial sources |
| | NKIbetaB | |
| Gp100 | Antibody or fragment | WO2013165940 or US20130295007 |
| | thereof | |
| Tyrosinase | Antibody or fragment | U.S. Pat. No. 5,843,674; or U.S. Ser. No. |
| | thereof | 19/950,504048 |
| EphA2 | Antibody or fragment | Yu et al., Mol Ther 22(1): 102-111 |
| | thereof | (2014) |
| GD3 | Antibody or fragment | U.S. Pat. No. 7,253,263; U.S. Pat. No. |
| | thereof | 8,207,308: US 20120276046; |
| | | EP1013761 A3; 20120276046; |
| | | WO2005035577; or U.S. Pat No. |
| | | 6,437,098 |
| Fucosyl GM1 | Antibody or fragment | US20100297138; or WO2007/067992 |
| | thereof | |
| sLe | antibody G193 (for | Scott A M et al., Cancer Res. 60: |
| | lewis Y) | 3254-61 (2000), also as described in |
| | | Neeson et al., J. Immunol. May 2013 |
| | | 190 (Meeting Abstract Supplement) |
| | | 177.10 |
| GM3 | antibody CA 2523449 | Commercial sources |
| | (mAb 14F7) | |
| HMWMAA | mAb9.2.27 | Kmiecik et al., Oncoimmunology |
| | | 3(1): e27185 (2014) (PMID: 24575382) |
| HMWMAA | Antibody or fragment | U.S. Pat. No. 6,528,481; |
| | thereof | WO2010033866; or US 20140004124 |
| o-acetyl-GD2 | antibody 8B6 | Commercial sources |
| TEMI/CD248 | Antibody or fragment | Marty et al., Cancer Lett. 235(2):298- |
| | thereof | 308 (2006); Zhao et al., J. Immunol. |
| | | Methods 363(2): 221-232 (2011) |
| CLDN6 | antibody IMAB027 | Ganymed Pharmaceuticals; |
| | | clinicaltrial.gov/show/NCT02054351. |
| TSHR | Antibody or fragment | U.S. Pat. No. 8,603,466; U.S. Pat. No. |
| | thereof | 8,501,415; or U.S. Pat. No. 8,309,693 |
| GPRCSD | antibody FAB6300A | R&D Systems |
| GPRC5D | Antibody LS-A4180 | Lifespan Biosciences |
| CD97 | Antibody or fragment | U.S. Pat. No. 6,846,911; de Groot et |
| | thereof | al., J. Immunol. 183(6): 4127-4134 |
| | | (2009) |
| CD97 | MAB3734 | R&D |
| ALK | Antibody or fragment | Mino-Kenudson et al., Clin. Cancer |
| | thereof | Res. 16(5): 1561-1571 (2010) |
| Polysialic acid | Antibody or fragment | Nagae et al., J. Biol. Chem. |
| | thereof | 288(47): 33784-33796 (2013) |
| PLAC1 | Antibody or fragment | Ghods et al., Biotechnol. Appl. |
| | thereof | Biochem. 2013 doi: 10.1002/bab. 1177 |
| GloboH | Antibody or fragment | Kudryashov V et al., Glycoconj |
| | thereof | 3.15(3): 243-9 (1998), Lou et al., Proc. |
| | | Natl. Acad. Sci. USA. 111(7): 2482- |
| | | 2487 (2014) |
| GloboH | Antibody MBr1 | Bremer E-G et al., J. Biol. Chem. |
| | | 259: 14773-14777 (1984) |
| NY-BR-1 | Antibody or fragment | Jager et al., Appl. Immunohistochem. |
| | thereof | Mol. Morphol. 15(1): 77-83 (2007) |
| WT-1 | Antibody 176ra33 | Dao et al., Sci. Transl. Med. 5(176) |
| | | (2013) |
| WT-1 | Antibody or fragment | WO2012/135854 |
| | thereof | |
| MAGE-A1 | Antibody or fragment | Willemsen et al., J. Immunol. |
| | thereof | 174(12): 7853-7858 (2005) |
| Sperm protein 17 | Antibody or fragment | Song et al., Target Oncol 2013 Aug. |
| | thereof | 14 (PMID: 23943313); Song et al., |
| | | Med Oncol 29(4): 2923-2931 (2012) |
| Tie2 | Antibody AB33 | Cell Signaling Technology |
| MAD-CT-2 | Antibody or fragment | PMID: 2450952; U.S. Pat. No. |
| | thereof | 7,635,753 |
| Fos-related antigen 1 | Antibody 12F9 | Novus Biologicals |
| MelanA/MART1 | Antibody or fragment | EP2514766 A2; or U.S. Pat. No. |
| | thereof | 7,749,719 |
| Sarcoma | Antibody or fragment | Luo et al., EMBO Mol. Med. |
| translocation | thereof | 4(6): 453-461 (2012) |
| breakpoints | | |
| TRP-2 | Antibody or fragment | Wang et al., J. Exp. Med. 184(6): 2207- |
| | thereof | 16 (1996) |
| CYP1B1 | Antibody or fragment | Maecker et al., Blood 102 (9): 3287- |
| | thereof | 3294 (2003) |
| RAGE-1 | Antibody MAB5328 | EMD Millipore |
| Human telomerase | Antibody cat no: LS- | Lifespan Biosciences |
| reverse transcriptase | B95-100 | |
| Intestinal carboxyl | Antibody 4F 12: cat no: | Lifespan Biosciences |
| esterase | LS-B6190-50 | |
| Mut hsp 70-2 | Monoclonal: cat no: | Lifespan Biosciences |
| | LS-C133261-100 | |
| FCAR | CD89/FCARAntibody | Sino Biological Inc. |
| | (Catalog#10414-H08H) | |
| CD300LF | Anti-CMRF35-like | BioLegend |
| | molecule 1 antibody, | |
| | Monoclonal[UP-D2] | |
| CD30OLF | Anti-CMRF35-like | R&D Systems |
| | molecule 1 antibody, | |
| | Monoclonal[234903] | |
| BST2 (also known as | Anti-CD317 antibody, | Antibodies-Online |
| CD317) | Monoclonal[3H4] | |
| BST2 (also known as | Anti-CD317 antibody, | R&D Systems |
| CD317) | Monoclonal[696739] | |
| EMR2 (also known | Anti-CD312 antibody, | Lifespan Biosciences |
| as CD312) | Monoclonal[LS-B8033] | |
| EMR2 (also known | Anti-CD312 antibody, | R&D Systems |
| as CD312) | Monoclonal[494025] | |
| GPC3 | Antibody hGC33 | Nakano K, Ishiguro T, Konishi H, et al. |
| | | Generation of a humanized anti-glypican 3 |
| | | antibody by CDR grafting and stability |
| | | optimization. Anticancer Drugs. 2010 |
| | | November; 21(10): 907-916 |
| GPC3 | Antibody MDX-1414, | Feng et al., “Glypican-3 antibodies: a new |
| | HN3, or YP7 | therapeutic target for liver cancer.” FEBS |
| | | Lett. 2014 Jan. 21; 588(2): 377-82 |
| CD38 | Monoclonal | Miltenyi Biotec |
| | recombinant REA572. | |
| | REA671 | |
| CD38 | IgG1κ human | Commercial sources |
| | monoclonal antibody | |
| | daratumumab | |
| CS-1 | Humanized | Tai et al., Blood 112(4): 1329-1337 (2008) |
| | monoclonal antibody | |
| | elotuzumab/HuLuc63 | |
| CD138 | Indatuximab | Biotest AG Dreieich, Germany; Kelly et |
| | Ravtansine (BT-062) | al., Blood 124(21): 4736 (2014) |
| PDGFRα | Olaratumab | Commercial sources |
| BCMA | Human monoclonal | Miltenyi Biotec |
| | recombinant antibody | |
| | REA315 | |

### Pro-Antigen

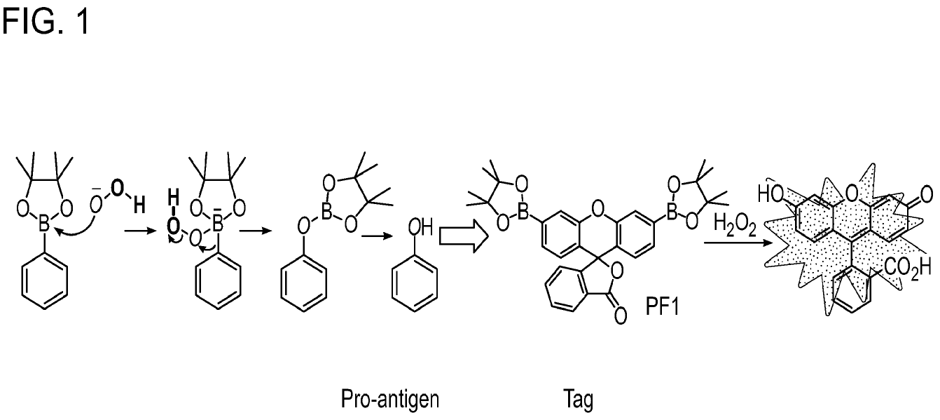

The pro-antigens of the present invention are small molecules that contain a boronic ester protecting group, and which become deprotected in the presence of ROS or RNS (hereinafter “ROS/RNS) such as hydrogen peroxide and peroxynitrite, such as by a hydrolysis reaction. The pro-antigen, which is also referred to herein as a “masked” or “caged” small molecule or tag, is inert in the sense that it does not bind and thus activate the CAR-T cells unless it becomes deprotected by ROS/RNS (FIG. 1).

The term “tag” or “recognition domain” is referred to herein as an “antigen small molecule” or “small molecule”. In general, a “small molecule” is understood in the art to be an organic molecule that is less than about 5 kilodaltons (kD) in size. In some embodiments, the small molecule is less than about 4 kD, about 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol.

The tag or recognition domain serves as the target for a universal CAR T cell. The recognition domain is linked to the targeting moiety in such a manner as not to interfere with the ability of the targeting moiety to bind to its ligand, e.g. a TAA. The recognition domain is one or more (i.e., plurality) small molecules. The small molecule may be synthetic or naturally-occurring. The small molecule may be biologically active or inactive.

In certain of these embodiments, the pro-antigen is a boronic ester of a small molecule such as an anthracene (e.g., ethonafide), a fluorescein (e.g., fluorescein or eosin), a rhodamine (e.g., rhodamine 6G), a rhodol, an acridine (e.g., acridine carboxamide), a xanthene (e.g., oftasceine, propantheline, or phloxine B), a pyrazine (e.g., bortezomi, pyrazimanide, or sitagliptin), a benzodiazepine (e.g., oxazepam or lorazepam), an opioid (e.g., fentanyl, morphine, oxycodone, or dextromethorphan), a cannabinoid (e.g., tetrahydrocannabinol (THC)), a tricyclic antidepressant (e.g., imipramine), benzoylecgonine, abuprenorphine, amphetamine, methamphetamine, meprobamate, tramadol, zolpidem, ketamine, lysergic acid diethylamide (LSD), phencyclidine (PCP), 3,4-methylenedioxy methamphetamine (MDMA), methadone, methaqualone, propoxyphene or norketimine.

Representative examples of fluoresceins that can be masked with a boronic ester and be linked to the targeting moieties include 5-carboxyfluorescein, 6-carboxyfluorescein, 5-(iodoacetamido)fluorescein, 5-([4,6-dichlorotriazin-2-yl]amino)fluorescein hydrochloride, 5-(bromomethyl)fluorescein, fluorescein 5(6)-isothiocyanate, and fluorescein 5-carbamoylmethylthiopropanoic acid.

Representative examples of anthracenes that can be masked with a boronic ester and be linked to the targeting moieties include anthraquinone, anthraquinone-2-carboxylate, 2-aminoanthraquinone, 2-iodoanthraquinone, 2-chloroanthraquinone, 2-bromoanthraquinone, 2-ethynylanthraquinone, 2-cyanoanthraquinone, anthraquinone-2-sulfonate, anthraquinone-2-carbonyl chloride and 2-hydroxyanthraquinone.

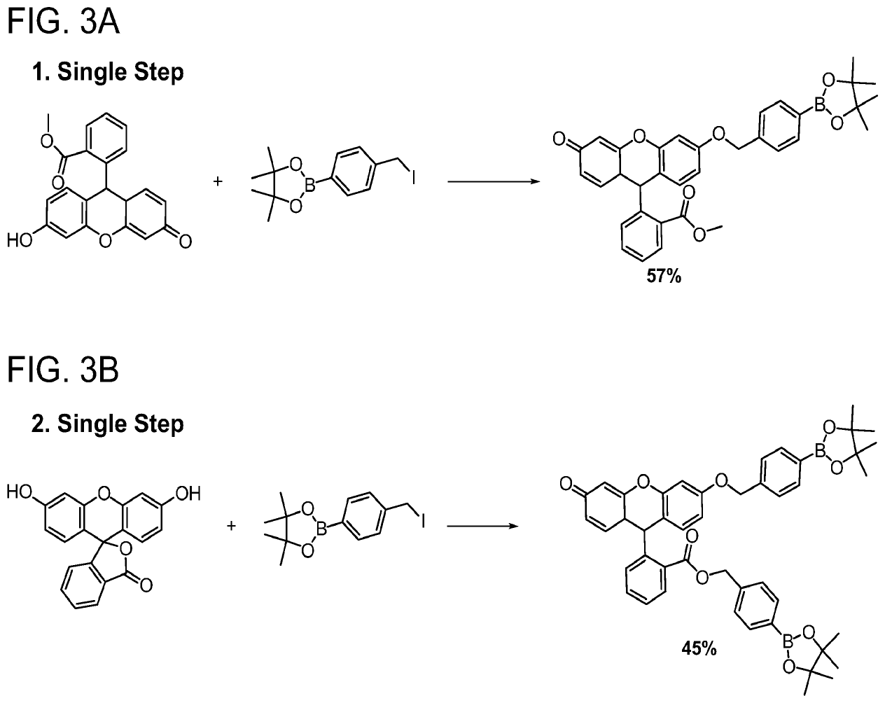

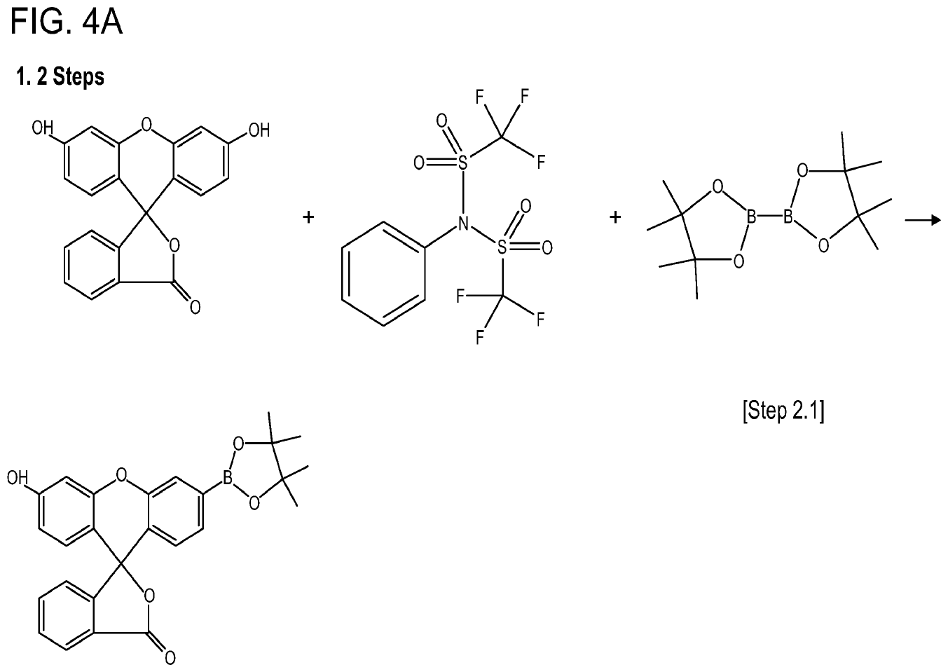

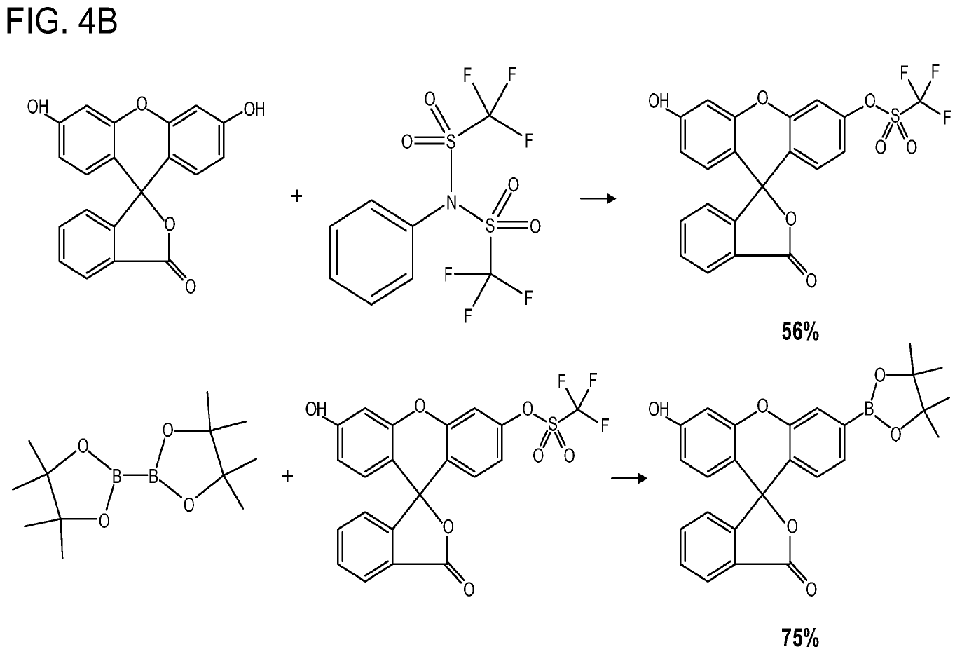

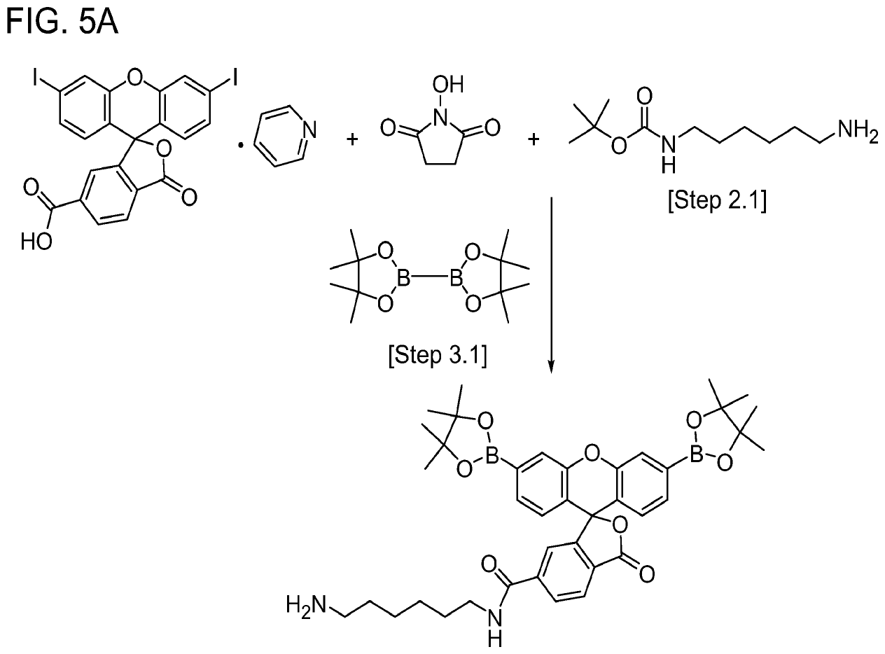

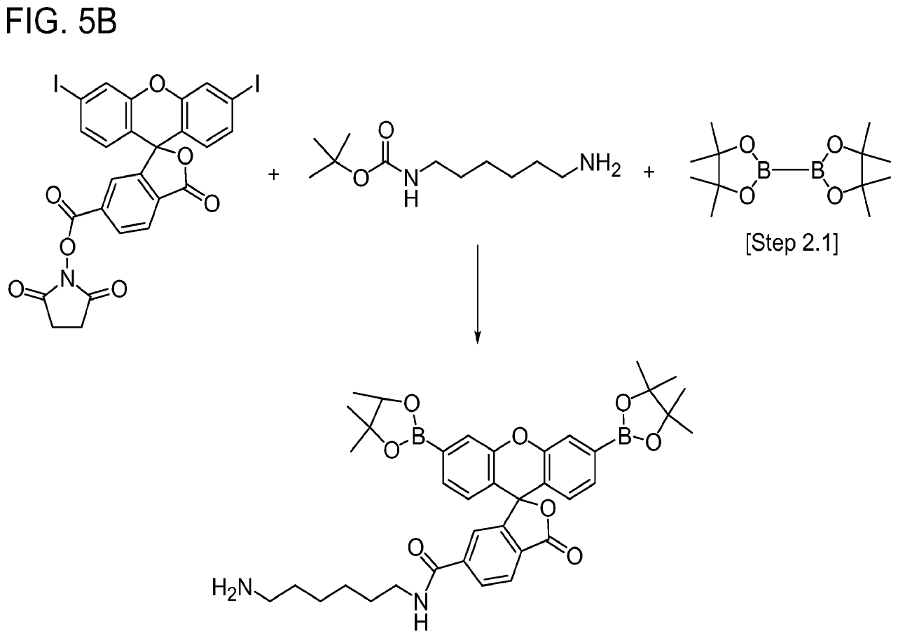

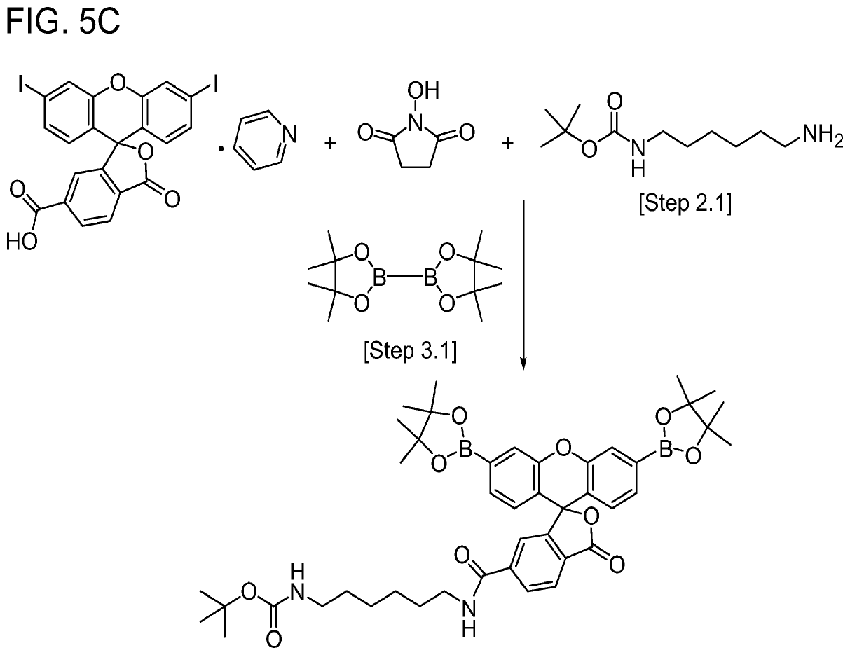

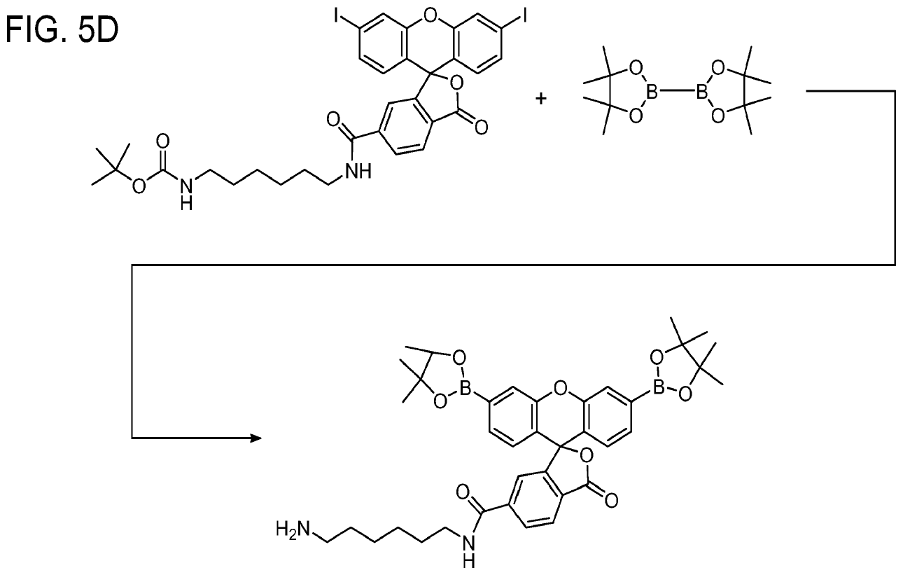

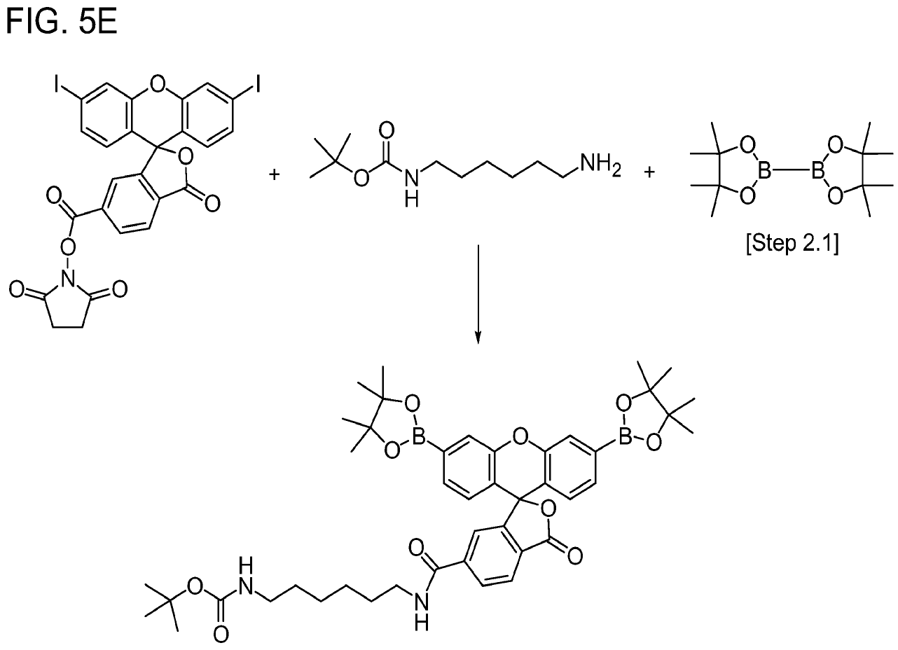

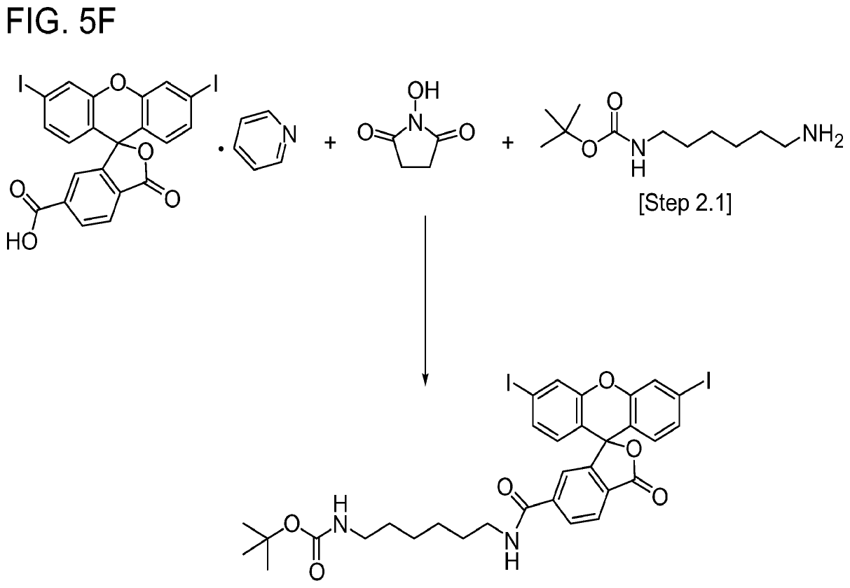

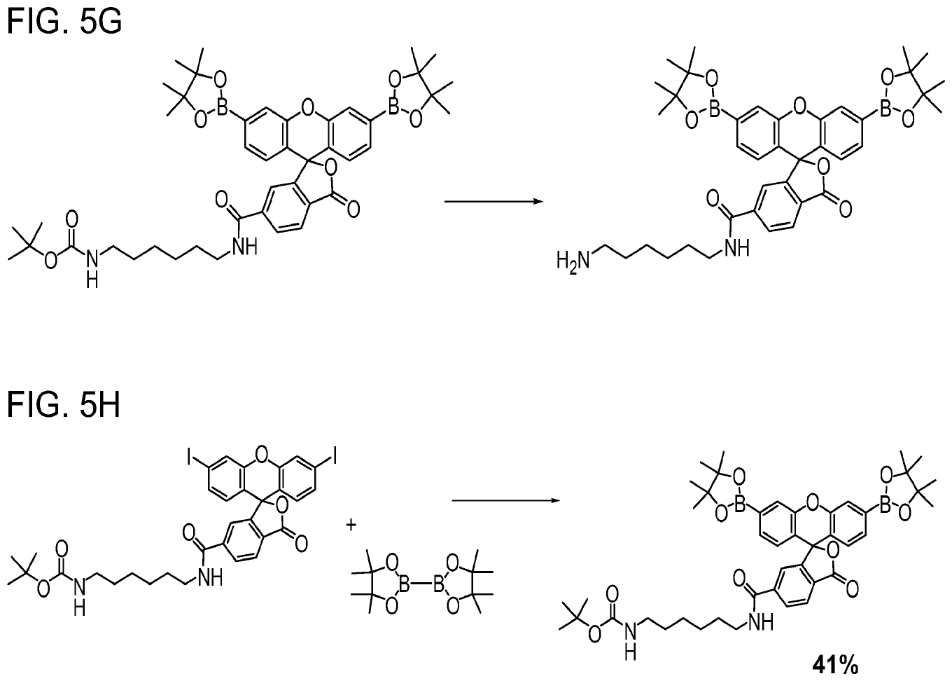

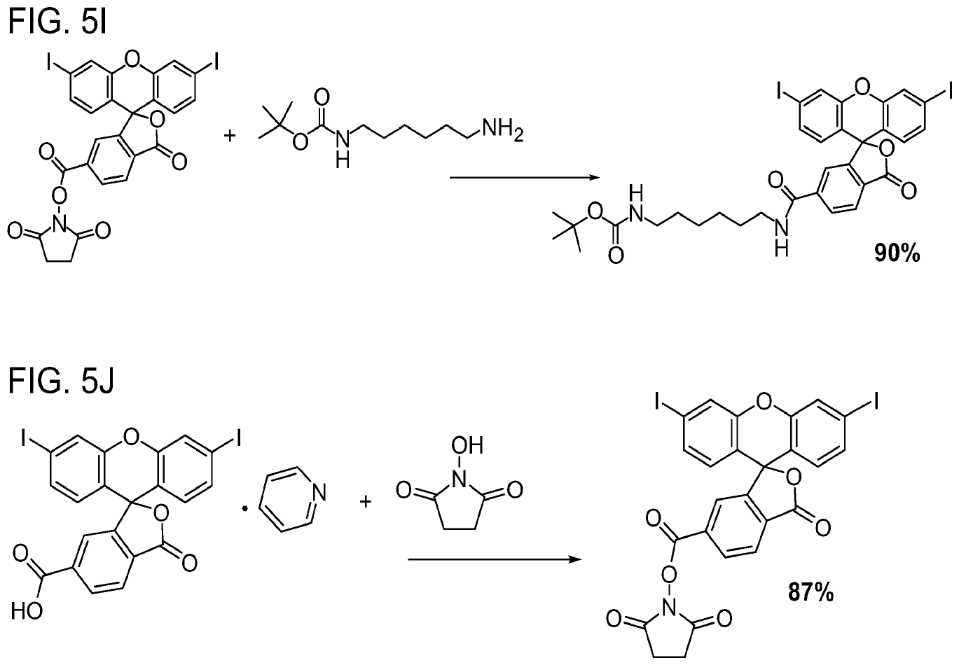

Pro-antigens of the present invention may be synthesized in accordance with methods known in the art. See, e.g., Lin et al., Meth. Enzymol. 526:19-43 (2013) (and publications referenced therein); Chang et al., J. Am. Chem. Soc. 126(47); 15392-3 (2004) (and publications referenced therein); Dickinson et al., J. Am. Chem. Soc. 132(16):5906-15 (2010) (and publications referenced therein). Additional details of the synthesis of pro-antigens as shown in FIG. 3A and FIG. 3B are disclosed in Debowska et al., Chemical Research in Toxicology 29(5):735-46 (2016). Additional details of the synthesis of pro-antigens as shown in FIG. 4A and FIG. 4B are disclosed in Rios et al, Free Radical Biology & Medicine 101:284-95 (2016). Additional details of the synthesis of pro-antigens as shown in FIG. 5A-FIG. 5J are disclosed in Wang et al., ACS Applied Materials & Interfaces 7(43):24110-18 (2015).

In some embodiments, the pro-antigen has a structure represented by (A) or (A′):

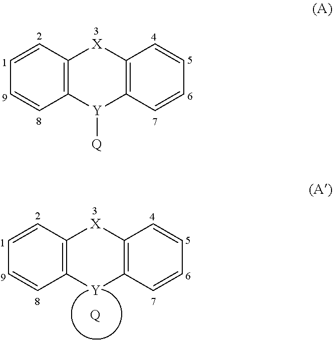

wherein X is C or O, Y is C, the boronic ester protecting group is present at one or more of positions 1-9 and Q represents one or more optionally substituted rings or a boronic ester protecting group, or a stereoisomer thereof. The optionally substituted rings are 4-7 carbocyclic or heterocyclic rings or a fused ring system, which may be saturated or non-saturated, and wherein heteroatoms may be selected from N, O and S.

In some embodiments, the bifunctional compound has a structure represented by formula (I):

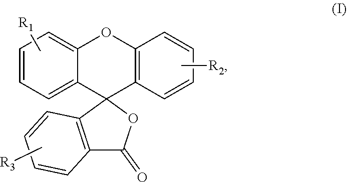

wherein R₁ is O, OH or a boronic ester protecting group:
R₂ is O, OH or a boronic ester protecting group; provided that at least one of R₁ and R₂ is a boronic ester protecting group; and

R₃ is

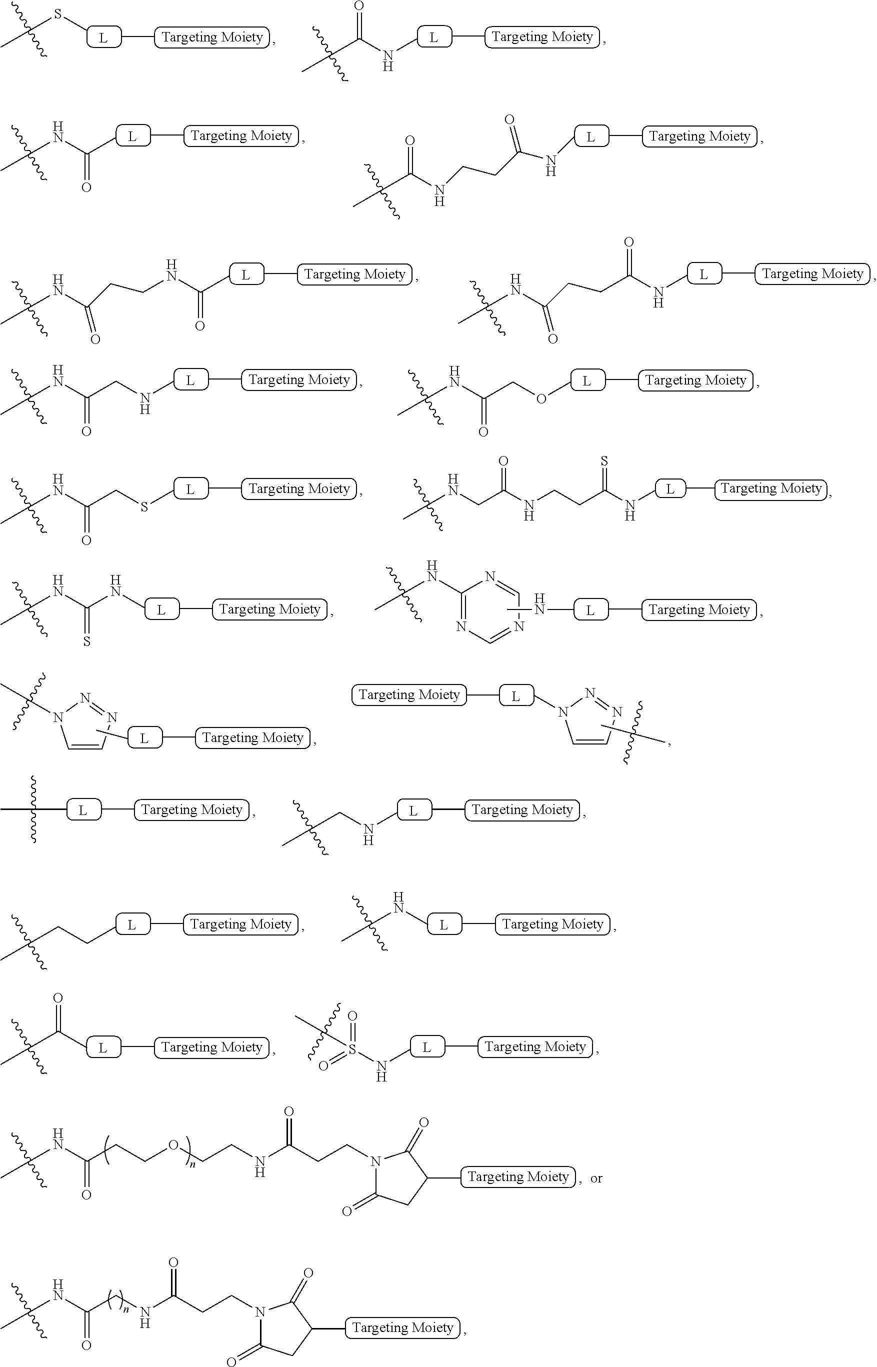

wherein

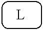

is absent or a linker and n is 1-12: wherein the boronic ester protecting group is

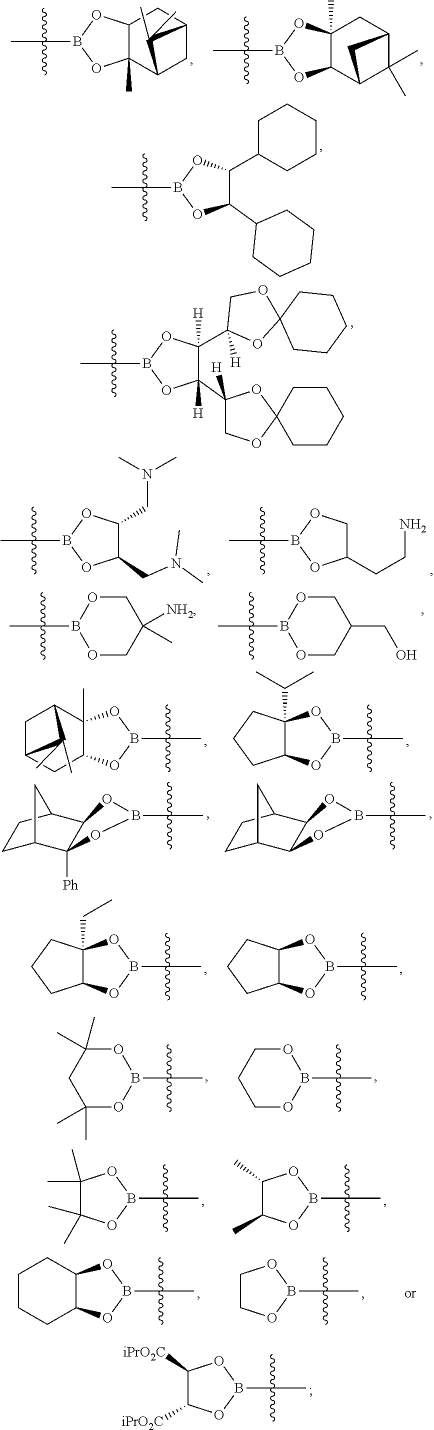

or stereoisomers thereof.

In some embodiments, the bifunctional compound has a structure represented by formula (Ia):

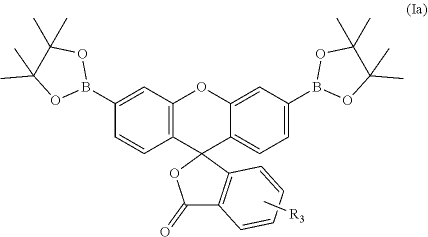

or a stereoisomer thereof.

In some embodiments, the bifunctional compound has a structure represented by formula (Ib):

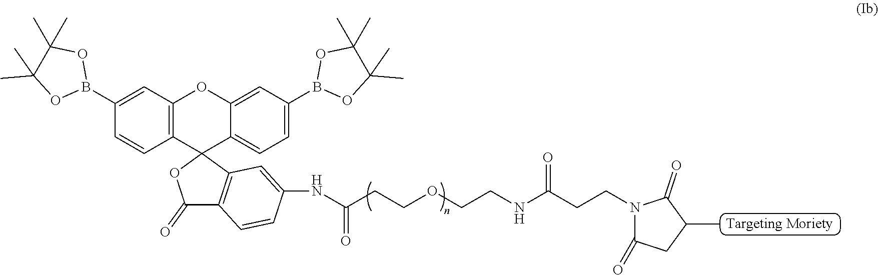

or stereoisomers thereof.

In some embodiments, the bifunctional compound has a structure represented by formula (Ic):

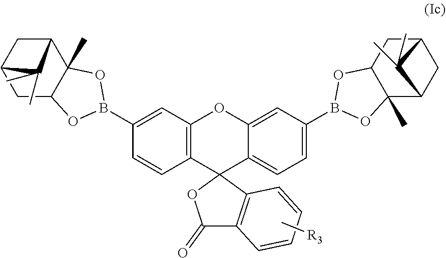

or a stereoisomer thereof.

In some embodiments, the bifunctional compound has a structure represented by formula (Id):

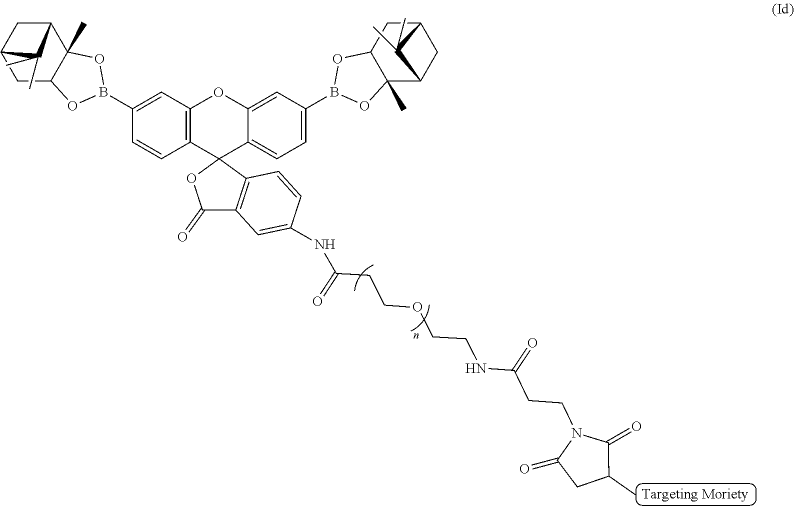

or a stereoisomer thereof.

In some embodiments, the bifunctional compound of formula (Id) is represented by the following structures:

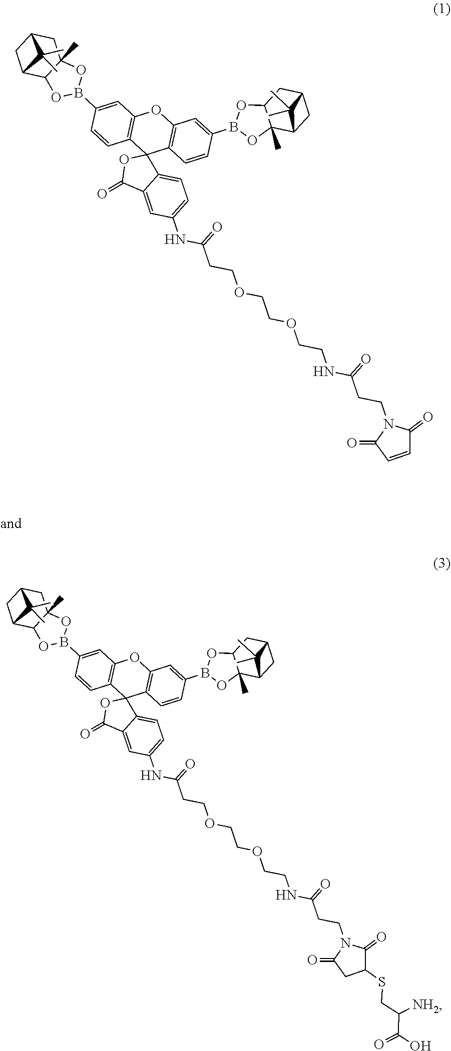

or a stereoisomer thereof.

In some embodiments the bifunctional compound has a structure represented by formula (II):

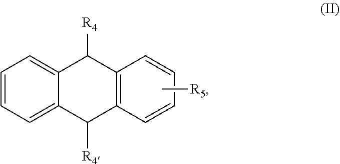

wherein each of R₄ and R_{4′} is independently O or a boronic ester protecting group; provided that at least one of R₄ and R_{4′} is a boronic ester protecting group; and

### R₅ is

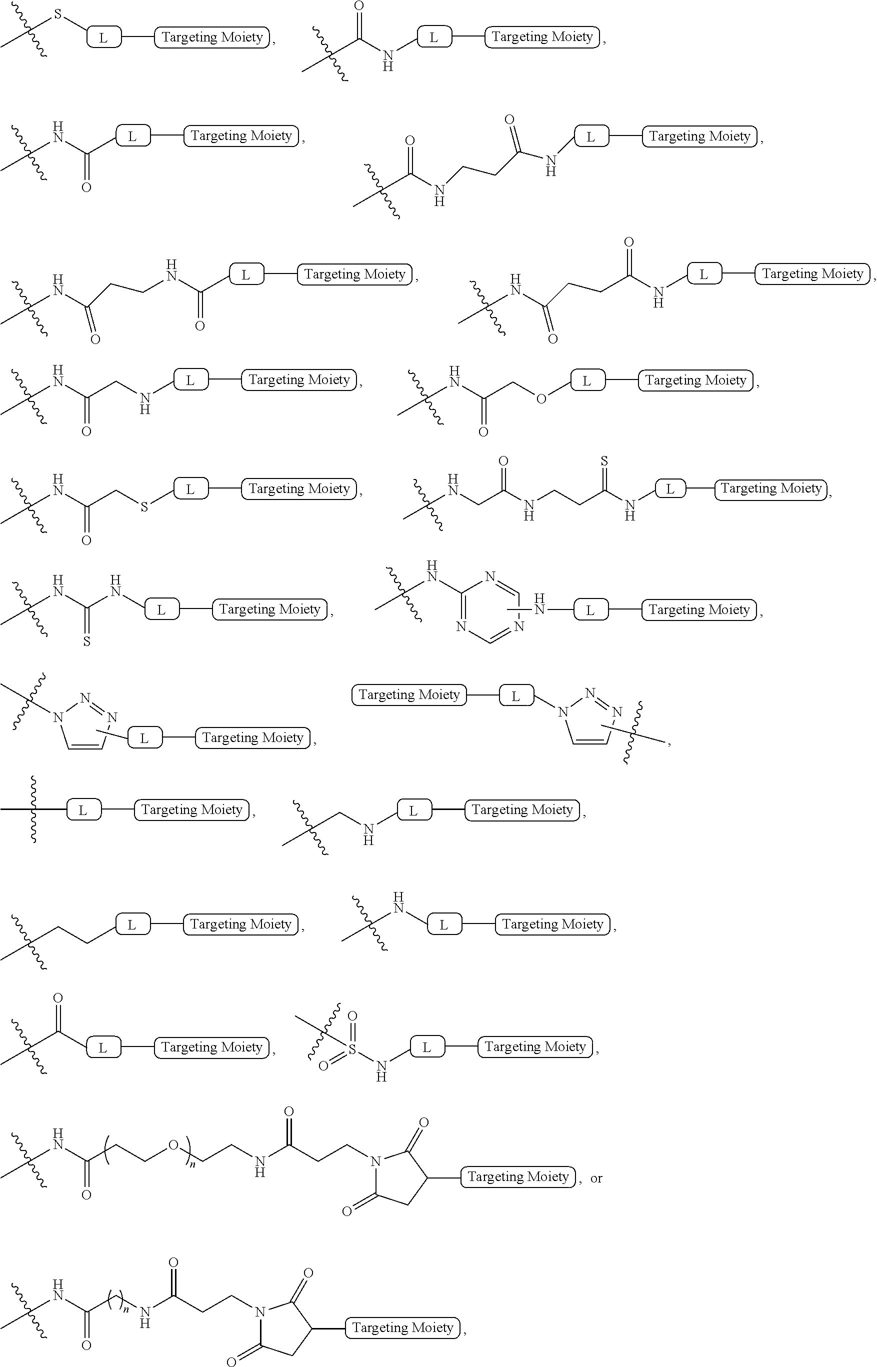

wherein

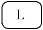

is absent or a linker and n is 1-12; wherein the boronic ester protecting group is

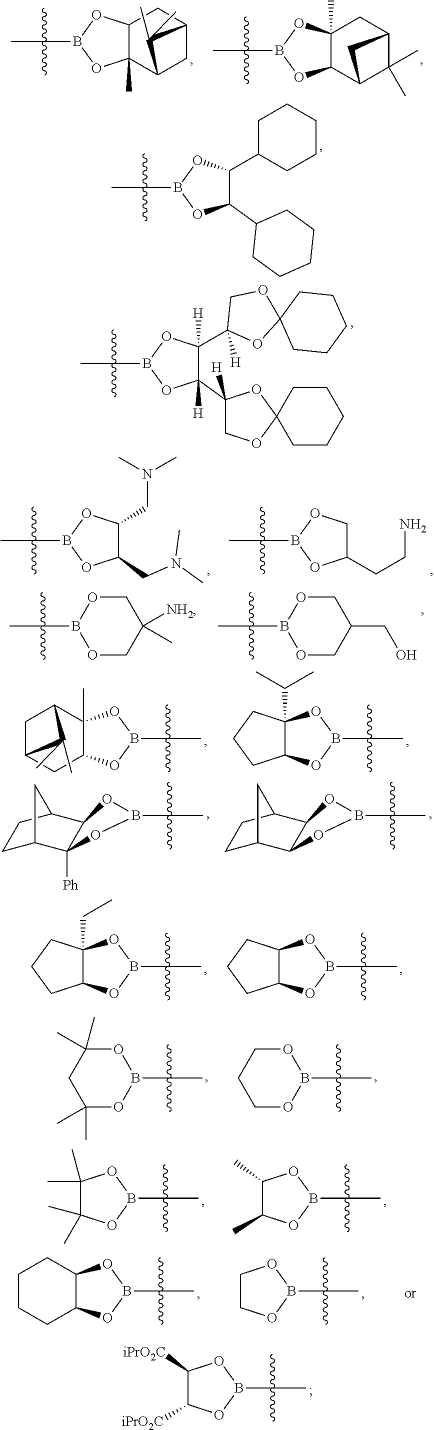

or a stereoisomer thereof.

In some embodiments, each of R₄ and R_{4′} is a boronic ester protecting group. In some embodiments, the bifunctional compound has a structure represented by formula (IIa):

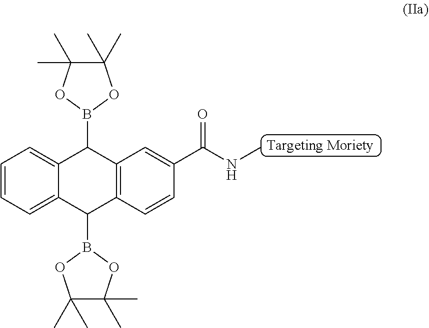 [SMILES: CNC(=O)c1ccc2c(c1)C(B1OC(C)(C)C(C)(C)O1)c1ccccc1C2B1OC(C)(C)C(C)(C)O1]

or stereoisomers thereof.

In some embodiments, the bifunctional compound has a structure represented by formula (IIb):

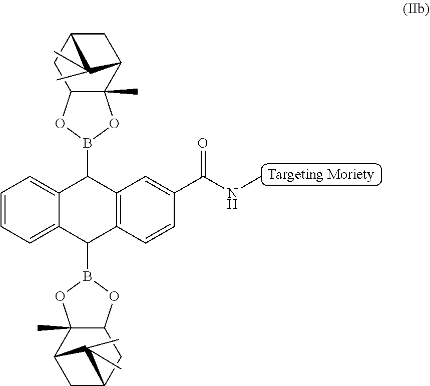 [SMILES: CNC(=O)c1ccc2c(c1)C(B1OC3C[C@@H]4C[C@@H](C4(C)C)[C@]3(C)O1)c1ccccc1C2B1OC2C[C@@H]3C[C@@H](C3(C)C)[C@]2(C)O1]

or a stereoisomer thereof.

Compounds of the present application may be in the form of a stereoisomer, which as used herein, embraces all isomers of individual compounds that differ only in the orientation of their atoms in space. The term stereoisomer includes mirror image isomers (enantiomers) of compounds, mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). Thus, the compounds of the present application may be in the form of individual isomers and substantially free of other isomers, and in the form of a mixture of various isomers, e.g., racemic mixtures of stereoisomers.

Boronic ester compounds of the present invention (the pro-antigens) may be synthesized in accordance with method known in the art. See. e.g., Roy and Brown, Monatshefte fur Chemie 138:879-87 (2007) and Bernardini et al., Chemistry Letters 38(7); 750-51 (2009), both of which are incorporated herein by reference.

Bifunctional compounds of the present invention may be synthesized in accordance with methods known in the art (FIG. 3A-FIG. 5J). See, e.g., WO2010/008519, incorporated herein by reference. In general, the small molecules of the invention possess or can be derivatized to possess chemical groups that react with primary amines (e.g., the N-terminal amine or lysines of polypeptides) or with sulfhydryl groups (e.g., cysteines of polypeptides), the polypeptides making up the targeting moieties of the bifunctional compounds. For example, the bifunctional compounds may be prepared by conjugating the targeting moiety to the pro-antigen using techniques such as chemical coupling and chemical cross-linkers. In some embodiments, the pro-antigen may be conjugated to the targeting moiety via a linker. Some of the factors that may make linkers necessary include: the necessity to recreate the microenvironment used to raise an antibody to the unmasked pro-antigen (i.e. the tag) which is used to engineer a CAR-T cell, the necessity to expose the small molecule to the solvent and make it accessible to the extracellular tag-binding domain of a CAR-T cell, and particularly hydrophobic small molecules may benefit from the use of a hydrophilic/polar linker e.g. polyethylene glycol (PEG). Linkers can vary in length, but shorter linkers are preferable because tags that are proximal to the target cell can better elicit a CAR-T cell response against the target cell. Representative examples of hydrophobic/nonpolar linkers include aliphatic linkers such as glycine, aminoheptanoic acid, aminohexanoic acid, aminopentanoic acid and aminotetranoic acid. Representative examples of polar linkers include polyethylene glycol moieties with for example, 1-12 repeating units (e.g., 2, 4, 6, 8, 10, or 12 repeating units).

For example, 6-aminofluorescein can be reacted with a maleimido-PEG-N-hydroxysuccinimide ester (NHS ester) to form compound (Ex-1) as shown in Scheme 1:

### Scheme 1:

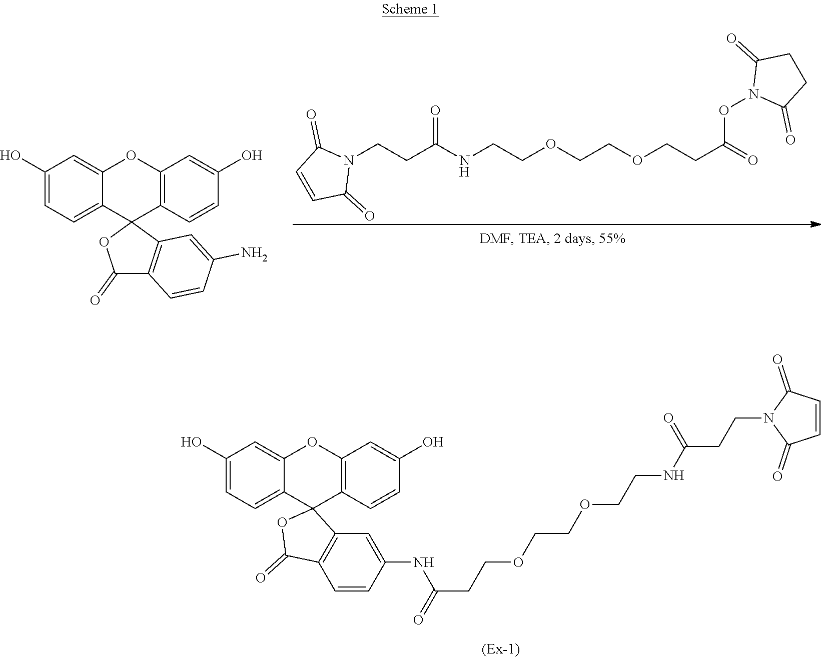 [SMILES: Nc1ccc2c(c1)C1(OC2=O)c2ccc(O)cc2Oc2cc(O)ccc21 ; O=C(CCN1C(=O)C=CC1=O)NCCOCCOCCC(=O)ON1C(=O)CCC1=O ; O=C(CCN1C(=O)C=CC1=O)NCCOCCOCCC(=O)Nc1ccc2c(c1)C1(OC2=O)c2ccc(O)cc2Oc2cc(O)ccc21]

The maleimide fluorescein derivative, compound (Ex-1 or 2), in turn reacts with sulfhydryl groups of targeting moieties to form a stable conjugate via a thioether bond as shown in Scheme 2:

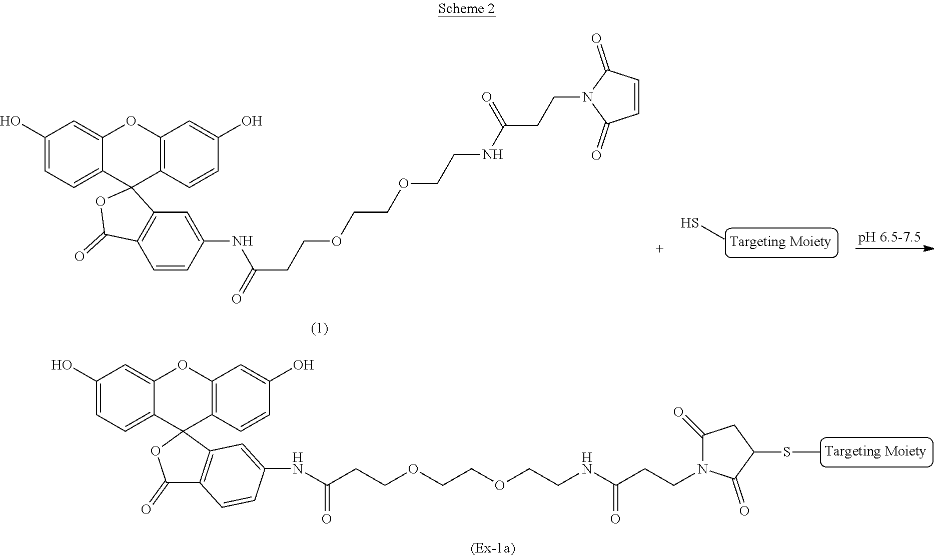

Representative examples of the synthesis of the bifunctional compounds having a masked pro-antigen are shown in Schemes 3 and 4:

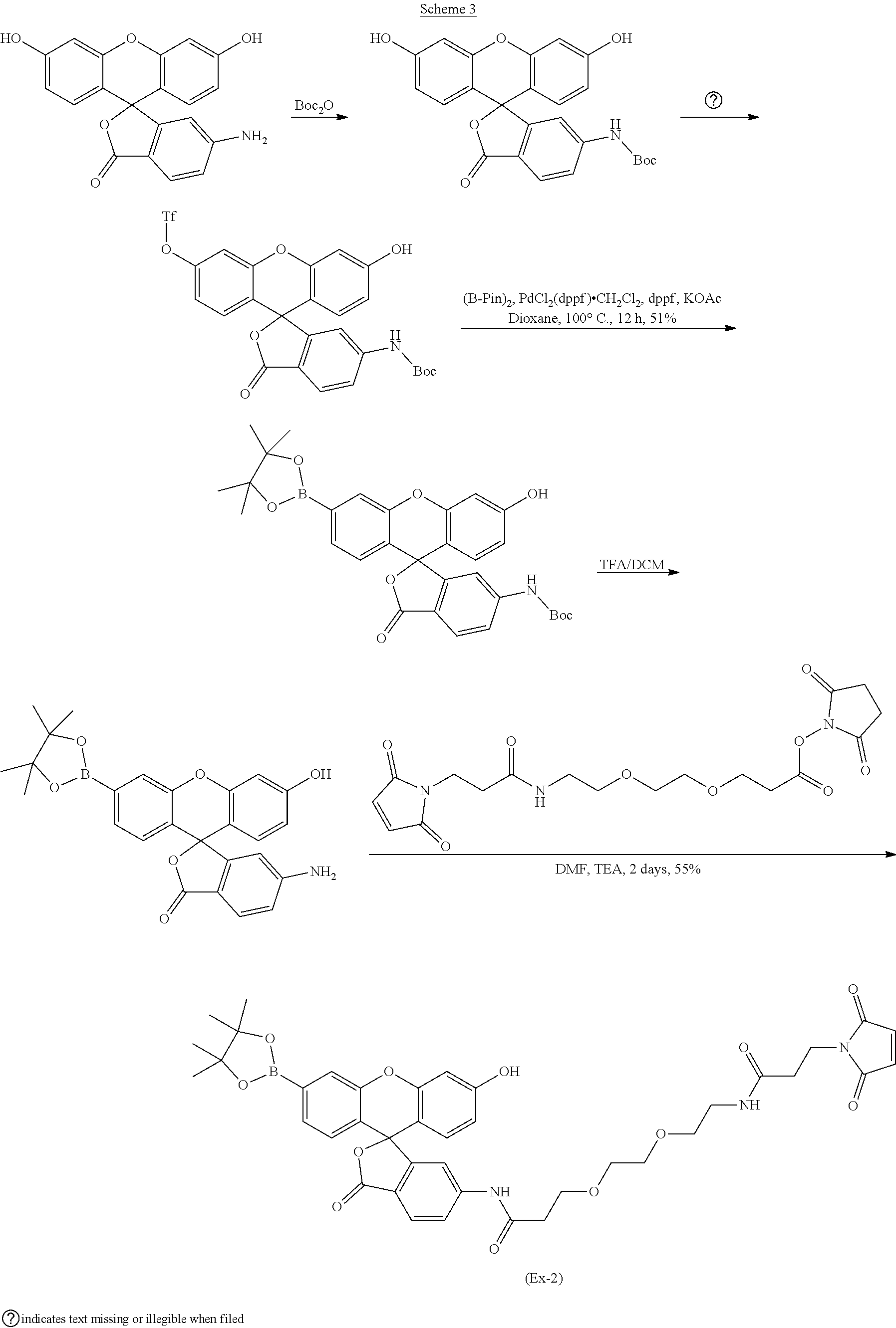

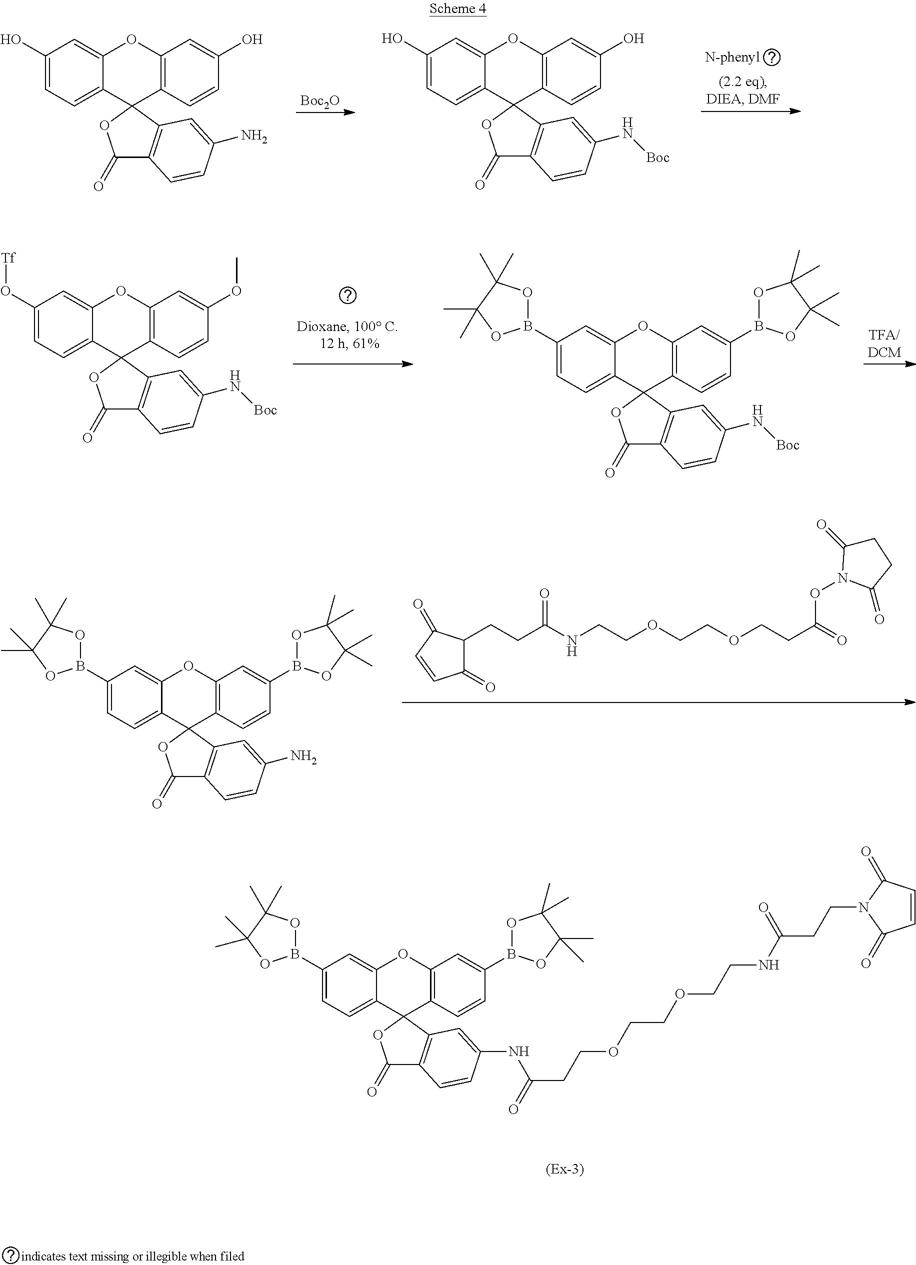

Each of compounds (2) and (3) can then be used in place of compound (1) as shown in Scheme 2 to generate the bifunctional compounds of the invention.

Another representative example of the synthesis of the bifunctional compounds having a masked pro-antigen using NHS-ester derivatives of fluorescein is shown in Scheme 5:

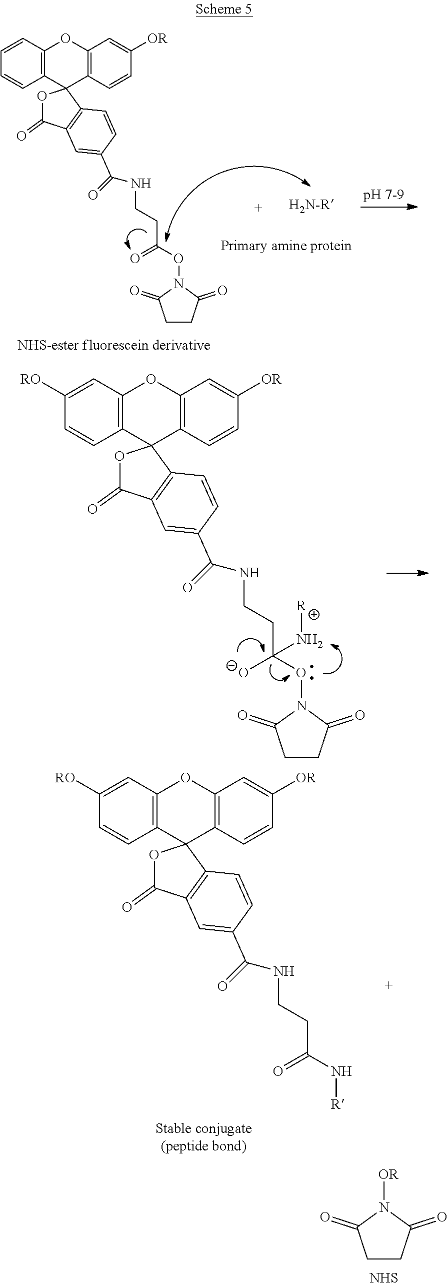

wherein OR is replaced with a boronic ester and R′ is a targeting moiety.

### CAR Effector Cells

Effector cells may be used in the methods of the present invention. The effector cells may be autologous, syngeneic or allogeneic, with the selection dependent on the disease to be treated and the means available to do so. Suitable populations of effector cells that may be used in the methods include any immune cells with cytolytic activity, such as T cells. Exemplary sub-populations of T cells include those expressing CD3′ such as CD3 CD8 T cells, CD3 CD4+ T cells, and NKT cells. Although in some embodiments the T cells are HLA-A2+ peripheral blood mononuclear cells (PBMC), they can be of any HLA background from PBMCs and utilized in an autologous, syngeneic or allogeneic system. T cells may also be isolated from any source, including from a tumor explant of the subject being treated or intra-tumoral T cells of the subject being treated. For the sake of convenience, the effector cells are hereinafter referred to as T cells, but it should be understood that any reference herein to T cells, unless otherwise indicated, is a reference to all effector cell types as defined herein.

The genetically engineered T cells used in the present invention have binding specificity for a particular unmasked pro-antigen (also referred to herein as a tag) that is conjugated to a targeting moiety (such as an antibody or functional fragment thereof) that binds to a tumor-associated antigen. Additional features of the CAR may include an activation domain that induces efficient target lysis upon T cell binding and activation, and the ability to substitute or replace the scFv portion of the CAR with one having specificity to any one of the unmasked pro-antigens or tags of the present invention. Since the unmasked pro-antigen serves as the target for a CAR T cell that is engineered with an extracellular binding domain that specifically binds the unmasked pro-antigen, and thus indirectly to the TAA, the CAR-T cells of the invention may be referred to as universal CAR-T cells or Binary Activated T cells (BAT-CARs”).

The BAT-CAR polypeptides typically include three domains. The first domain is an extracellular ligand or a tag-binding domain: the second domain is transmembrane (TM) domain; and the third domain is the T cell activation domain.

### Extracellular Ligand

The first domain is typically present at the amino terminal end of the BAT-CAR polypeptide, and thus external to the T cell, which permits the tag-binding domain unfettered access to the tagged protein that is bound to the target cell. The tag-binding domain is typically an antibody or an antigen-binding fragment thereof. In some embodiments, the antibodies are human or humanized antibodies, or antigen-binding fragments thereof.

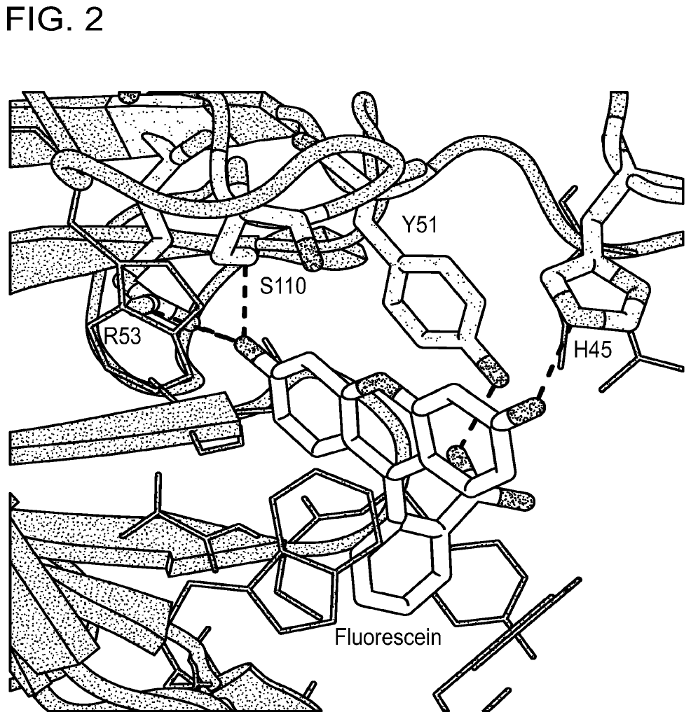

The tag-binding domain is designed to specifically bind the unmasked pro-antigen that is covalently linked to the targeting moiety that binds the target cells (e.g., the cancer cells). For example, when the tag or unmasked pro-antigen is fluorescein or a fluorescein derivative, the tag-binding domain specifically binds fluorescein or a fluorescein derivative but not the caged molecule (FIG. 2), examples of which are known in the art, e.g., 4M5.3 ScFv, disclosed in Midelfort et al. J. Mol. Biol. 343:685-701 (2004).

The type of antibody is not critical; it may also be polyclonal, monoclonal, chimeric or humanized. The antibodies may be obtained from any species of animal, e.g., a human, simian, mouse, rat, rabbit, guinea pig, horse, cow, sheep, goat, pig, dog or cat. Nor is there a limitation on the particular class of antibody that may be used, including IgG₁, IgG₂, IgG₃, IgG₄, IgM, IgA₁, IgA₂, IgD and IgE antibodies. Antibody fragments, which also may be used, include single-chain variable fragment (scFv), single chain antibodies. F(ab′)₂ fragments. Fab fragments, and fragments produced by a Fab expression library, provided that the antibody fragments retain the ability to bind the selected tag.

BAT-CARs of the present invention may be produced using commercially-available extracellular ligands, at least to the extent that the unmasked pro-antigens are known. Alternatively, antibodies and fragments thereof that specifically bind the unmasked pro-antigen can be prepared using standard techniques, e.g., continuous cell lines in culture for monoclonal antibody production. Representative techniques include the hybridoma technique originally described by Koehler and Milstein (Nature 256:495-497 (1975)), the human B-cell hybridoma technique (Kosbor et al., Immunol Today 4:72 (1983); Cote et al., Proc Natl. Acad. Sci 80:2026-2030 (1983)), and the EBV-hybridoma technique (Cole et al., Monoclonal Antibodies and Cancer Therapy, Alan R. Liss Inc. New York N.Y., pp 77-96 (1985)). Techniques developed for the production of “chimeric antibodies.” i.e., the splicing of mouse antibody genes to human antibody genes to obtain a molecule with appropriate antigen specificity and biological activity, can also be used (Morrison et al., Proc Natl. Acad. Sci 81:6851-6855 (1984); Neuberger et al., Nature 312:604-608(1984); Takeda et al., Nature 314:452-454 (1985)). As known in the art, a humanized antibody or antibody fragment has one or more amino acid residues, typically from a variable domain of an antibody from a nonhuman source. Humanized antibodies or antibody fragments may contain one or more CDRs from nonhuman immunoglobulin molecules, and framework regions that are derived completely or mostly from human germline. Techniques for humanizing antibodies or antibody fragments are well known, and include CDR grafting, veneering or resurfacing, and chain shuffling. See, also, Jones et al., Nature 321:522-525 (1986); Riechmann et al., Nature 332:323-327 (1988); Verhoeven et al., Science 239:1534-1536 (1988)).

In some embodiments, the tag-binding domain of the BAT-CAR-T is a single-chain variable fragment (scFv). A scFv includes the variable regions of the heavy (VH) and light chains (VL) of an antibody, and typically includes up to about 50, e.g., about 10 amino acid residues. The linker can either connect the N-terminus of the VH with the C-terminus of the VL, or vice versa. ScFvs can be prepared according to methods known in the art (see, e.g., Bird et al., Science 242:423-426 (1988) and Huston et al., Proc. Natl. Acad. Sci. USA 85:5879-5883 (1988)). In some embodiments, the linker sequence includes amino acids glycine and serine, and in some cases, sets of glycine and serine repeats such as (Gly₄Ser)ₙ, where n is an integer equal to or greater than 1. The length and amino acid composition of the linker may be varied e.g., to achieve optimal folding and interaction between the VH and VL to create a functional epitope. See, e.g., Hollinger et al., Proc Natl Acad. Sci. U.S.A. 90:6444-6448 (1993).

Other types of antibody fragments having specificity for unmasked pro-antigens that may be useful in the present invention include Fv, Fab, and (Fab′)₂ fragments. See, e.g., U.S. Pat. No. 4,946,788.

The second domain is a transmembrane (TM) domain, which allows the BAT-CAR to be anchored into the cell membrane of the T cell. The BAT-CAR can be designed to include a transmembrane domain that is attached to the extracellular domain of the CAR. The transmembrane domain may be derived from the same protein or from a different protein from which the other domains of the CAR (e.g., signaling domain, costimulatory domain and hinge domain) are derived. The transmembrane domain may be derived from a natural or from a recombinant source. Where the source is natural, the domain may be derived from any membrane-bound or transmembrane protein. Representative examples of transmembrane domains that may be useful in the present invention include the transmembrane regions of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154.

In some embodiments, the transmembrane domain is attached to the extracellular region of the CAR, e.g., the antigen binding domain of the CAR, via a hinge, such as a hinge from a human protein. Sources of hinge domains include human Ig (immunoglobulin) hinges (e.g., an IgG4 hinge, an IgD hinge), and a CD8 (e.g., CD8α hinge).

The third domain of the BAT-CAR is the T cell activation domain, also known as the intracellular signaling domain, which aids in T cell activation upon binding of the CAR to the tagged protein that is bound to the target cell. An intracellular signaling domain is generally responsible for activation of at least one of the normal effector functions of the effector cell in which the CAR has been introduced. Examples of intracellular signaling domains for use in the CAR of the invention include the cytoplasmic sequences of the T cell receptor (TCR) and co-receptors that act in concert to initiate signal transduction following antigen receptor engagement. Signals generated through the TCR alone are often insufficient for full activation of T cells; thus, a secondary or costimulatory signal is also generally required. Thus, T cell activation is mediated by two distinct classes of cytoplasmic signaling sequences, namely those that initiate antigen-dependent primary activation through the TCR (i.e., the primary intracellular signaling domains) and those that act in an antigen-independent manner to provide a secondary or costimulatory signal (i.e., the secondary cytoplasmic or costimulatory domain). The primary signaling domain regulates primary activation of the TCR complex either in a stimulatory way, or in an inhibitory way. Primary intracellular signaling domains that act in a stimulatory manner may contain signaling motifs known as immunoreceptor tyrosine-based activation motifs (ITAMs). Representative examples of ITAM-containing primary intracellular signaling domains that may be suitable for use in the present invention include those of CD3ζ, common FcRγ (FCER1G), Fc-γ RIIa, FcR-β (Fc-ε R1b), CD3γ, CD3δ, and CD3ε. In some embodiments, the BAT-CARs include an intracellular signaling domain that contains the primary signaling domain of CD3ζ.

The intracellular signaling domain of the BAT-CAR may also include at least one other intracellular signaling or co-stimulatory domain. A costimulatory molecule is a cell surface molecule other than an antigen receptor or its ligands that is required for an efficient response of lymphocytes to an antigen. Representative examples of co-stimulatory domains that may be useful in the BAT-CARs of the present invention include CD27, CD28, 4-1BB (CD137), OX40, CD30, CD40, PD-1, ICOS, HVEM (LIGHTR), lymphocyte function-associated antigen-1 (LFA-1), CD2, CD7, LIGHT, NKG2C, and B7-H3, CD27 co-stimulation, for example, has been demonstrated to enhance expansion, effector function, and survival of human CART cells in vitro and augments human T cell persistence and antitumor activity in vivo (Song et al., Blood 119(3):696-706 (2012)).

The intracellular signaling domain may be designed to include one or more, e.g., 1, 2, 3, 4, 5, or more costimulatory signaling domains, which may be linked to each other in a specified or random order, optionally via a linker molecule. Polypeptide linkers that are about 1-10 amino acids in length may join consecutive intracellular signaling sequences. Examples of such linkers include doublets such as Gly-Ser, and single amino acids, e.g., Ala and Gly. Combinations that may constitute the T-cell activation domain may be based on the cytoplasmic regions of CD28, CD137 (4-1BB). OX40 and HVEM, which serve to enhance T cell survival and proliferation; and CD3 CD3ζ and FcRε, which induce T cell activation. For example, CD3ζ, which contains 3 ITAMs, is the most commonly used intracellular domain component of CARS, transmits an activation signal to the T cell after antigen is bound. However, to provide additional co-stimulatory signaling, CD28 and OX40 domains can be used with CD3ζ which enable the BAT-CAR T cells to transmit the proliferative/survival signals.

A representative example of a polynucleotide that encodes an anti-FL CAR-CD28-4-1BB-CD3ζ has the sequence designated as SEQ ID NO:1:

(SEQ ID NO: 1)
ATGGCTCTGCCTGTGACAGCTCTGCTGCTGCCTCTGGCTCTGCTTCTGCA
TGCCGCCAGACCTGACGTGGTCATGACACAGACACCTCTGAGCCTGCCTG
TGTCTCTGGGAGATCAGGCCAGCATCAGCTGCAGATCTAGCCAGAGCCTG
GTGCACAGCAACGGCAACACCTACCTGCGGTGGTATCTGCAGAAGCCCGG
CCAGTCTCCTAAGGTGCTGATCTACAAGGTGTCCAACAGAGTGTCCGGCG
TGCCCGATAGATTTTCTGGCAGCGGCTCTGGCACCGACTTCACCCTGAAG
ATCAATAGAGTGGAAGCCGAGGACCTGGGCGTGTACTTCTGTAGCCAGTC
TACCCACGTGCCATGGACCTTTGGCGGCGGAACAAAGCTGGAAATCAAGA
GCAGCGCCGACGACGCCAAGAAGGACGCCGCTAAGAAGGATGACGCCAAA
AAAGACGATGCCAAAAAGGATGGCGGCGTGAAGCTGGACGAAACAGGCGG
AGGACTTGTTCAGCCTGGCGGAGCCATGAAGCTGAGCTGTGTGACCAGCG
GCTTCACCTTCGGCCACTACTGGATGAACTGGGTCCGACAGAGCCCTGAG
AAAGGCCTGGAATGGGTCGCCCAGTTCAGAAACAAGCCCTACAACTACGA
AACCTACTACAGCGACAGCGTGAAGGGCAGATTCACCATCAGCCGGGACG
ACAGCAAGTCCAGCGTGTACCTGCAGATGAACAACCTGCGCGTGGAAGAT
ACCGGCATCTACTACTGTACCGGCGCCAGCTACGGCATGGAATATCTCGG
CCAGGGCACCAGCGTGACCGTGTCTACAACAACCCCTGCTCCTCGGCCTC
CTACACCAGCTCCTACAATTGCCAGCCAGCCACTGTCTCTGAGGCCCGAA
GCTTGTAGACCTGCTGCAGGCGGAGCCGTGCATACAAGAGGACTGGATTT
CGCCTGCGACTTCTGGGTGCTCGTGGTTGTTGGCGGAGTGCTGGCTTGTT
ACTCCCTGCTGGTTACCGTGGCCTTCATCATCTTTTGGGTCCGAAGCAAG
CGGAGCCGGCTGCTGCACAGCGACTACATGAACATGACCCCTAGACGGCC
CGGACCTACCAGAAAGCACTACCAGCCTTACGCTCCTCCTAGAGACTTCG
CCGCCTACAGATCCAAGCGGGGCAGAAAGAAGCTGCTGTACATCTTCAAG
CAGCCCTTCATGCGGCCCGTGCAGACCACACAAGAGGAAGATGGCTGCTC
CTGCAGATTCCCCGAGGAAGAAGAAGGCGGCTGCGAGCTGAGAGTGAAGT
TCAGCAGATCCGCCGACGCTCCTGCCTATCAGCAGGGACAGAACCAGCTG
TACAACGAGCTGAACCTGGGGAGAAGAGAAGAGTACGACGTGCTGGACAA
GCGGAGAGGCAGAGATCCTGAGATGGGCGGAAAGCCCCAGCGGAGAAAGA
ATCCTCAAGAGGGCCTGTATAATGAGCTGCAGAAAGACAAGATGGCCGAG
GCCTACAGCGAGATCGGAATGAAGGGCGAGCGCAGAAGAGGCAAGGGACA
CGATGGACTGTACCAGGGCCTGAGCACCGCCACCAAGGATACCTATGATG
CCCTGCACATGCAGGCCCTGCCACCTAGATGATGA

A representative example of a polynucleotide that encodes an anti-FL CAR-4-1BB-CD3ζ has the sequence designated as SEQ ID NO:2:

ATGGCTCTGCCTGTGACAGCTCTGCTGCTGCCTCTGGCTCTGCTTCTGCA
TGCCGCCAGACCTGACGTGGTCATGACACAGACACCTCTGAGCCTGCCTG
TGTCTCTGGGAGATCAGGCCAGCATCAGCTGCAGATCTAGCCAGAGCCTG
GTGCACAGCAACGGCAACACCTACCTGCGGTGGTATCTGCAGAAGCCCGG
CCAGTCTCCTAAGGTGCTGATCTACAAGGTGTCCAACAGAGTGTCCGGCG
TGCCCGATAGATTTTCTGGCAGCGGCTCTGGCACCGACTTCACCCTGAAG
ATCAATAGAGTGGAAGCCGAGGACCTGGGCGTGTACTTCTGTAGCCAGTC
TACCCACGTGCCATGGACCTTTGGCGGCGGAACAAAGCTGGAAATCAAGA
GCAGCGCCGACGACGCCAAGAAGGACGCCGCTAAGAAGGATGACGCCAAA
AAAGACGATGCCAAAAAGGATGGCGGCGTGAAGCTGGACGAAACAGGCGG
AGGACTTGTTCAGCCTGGCGGAGCCATGAAGCTGAGCTGTGTGACCAGCG
GCTTCACCTTCGGCCACTACTGGATGAACTGGGTCCGACAGAGCCCTGAG
AAAGGCCTGGAATGGGTCGCCCAGTTCAGAAACAAGCCCTACAACTACGA
AACCTACTACAGCGACAGCGTGAAGGGCAGATTCACCATCAGCCGGGACG
ACAGCAAGTCCAGCGTGTACCTGCAGATGAACAACCTGCGCGTGGAAGAT
ACCGGCATCTACTACTGTACCGGCGCCAGCTACGGCATGGAATATCTCGG
CCAGGGCACCAGCGTGACCGTGTCTACAACAACCCCTGCTCCTCGGCCTC
CTACACCAGCTCCTACAATTGCCAGCCAGCCACTGTCTCTGAGGCCCGAA
GCTTGTAGACCTGCTGCAGGCGGAGCCGTGCATACAAGAGGACTGGATTT
CGCCTGCGACTTCTGGGTGCTCGTGGTTGTTGGCGGAGTGCTGGCTTGTT
ACTCCCTGCTGGTTACCGTGGCCTTCATCATCTTTTGGGTCAAGCGGGGC
AGAAAGAAGCTGCTGTACATCTTCAAGCAGCCCTTCATGCGGCCCGTGCA
GACCACACAAGAGGAAGATGGCTGCTCCTGCAGATTCCCCGAGGAAGAAG
AAGGCGGCTGCGAGCTGAGAGTGAAGTTCAGCAGATCCGCCGACGCTCCT
GCCTATCAGCAGGGACAGAACCAGCTGTACAACGAGCTGAACCTGGGGAG
AAGAGAAGAGTACGACGTGCTGGACAAGCGGAGAGGCAGAGATCCTGAGA
TGGGCGGAAAGCCCCAGCGGAGAAAGAATCCTCAAGAGGGCCTGTATAAT
GAGCTGCAGAAAGACAAGATGGCCGAGGCCTACAGCGAGATCGGAATGAA
GGGCGAGCGCAGAAGAGGCAAGGGACACGATGGACTGTACCAGGGCCTGA
GCACCGCCACCAAGGATACCTATGATGCCCTGCACATGCAGGCCCTGCCA
CCTAGATGATGA

A representative example of a polynucleotide that encodes an anti-FL CAR-CD28-CD3ζ has the sequence designated as SEQ ID NO:3:

ATGGCTCTGCCTGTGACAGCTCTGCTGCTGCCTCTGGCTCTGCTTCTGCA
TGCCGCCAGACCTGACGTGGTCATGACACAGACACCTCTGAGCCTGCCTG
TGTCTCTGGGAGATCAGGCCAGCATCAGCTGCAGATCTAGCCAGAGCCTG
GTGCACAGCAACGGCAACACCTACCTGCGGTGGTATCTGCAGAAGCCCGG
CCAGTCTCCTAAGGTGCTGATCTACAAGGTGTCCAACAGAGTGTCCGGCG
TGCCCGATAGATTTTCTGGCAGCGGCTCTGGCACCGACTTCACCCTGAAG
ATCAATAGAGTGGAAGCCGAGGACCTGGGCGTGTACTTCTGTAGCCAGTC
TACCCACGTGCCATGGACCTTTGGCGGCGGAACAAAGCTGGAAATCAAGA
GCAGCGCCGACGACGCCAAGAAGGACGCCGCTAAGAAGGATGACGCCAAA
AAAGACGATGCCAAAAAGGATGGCGGCGTGAAGCTGGACGAAACAGGCGG
AGGACTTGTTCAGCCTGGCGGAGCCATGAAGCTGAGCTGTGTGACCAGCG
GCTTCACCTTCGGCCACTACTGGATGAACTGGGTCCGACAGAGCCCTGAG
AAAGGCCTGGAATGGGTCGCCCAGTTCAGAAACAAGCCCTACAACTACGA
AACCTACTACAGCGACAGCGTGAAGGGCAGATTCACCATCAGCCGGGACG
ACAGCAAGTCCAGCGTGTACCTGCAGATGAACAACCTGCGCGTGGAAGAT
ACCGGCATCTACTACTGTACCGGCGCCAGCTACGGCATGGAATATCTCGG
CCAGGGCACCAGCGTGACCGTGTCTACAACAACCCCTGCTCCTCGGCCTC
CTACACCAGCTCCTACAATTGCCAGCCAGCCACTGTCTCTGAGGCCCGAA
GCTTGTAGACCTGCTGCAGGCGGAGCCGTGCATACAAGAGGACTGGATTT
CGCCTGCGACTTCTGGGTGCTCGTGGTTGTTGGCGGAGTGCTGGCTTGTT
ACTCCCTGCTGGTTACCGTGGCCTTCATCATCTTTTGGGTCCGAAGCAAG
CGGAGCCGGCTGCTGCACAGCGACTACATGAACATGACCCCTAGACGGCC
CGGACCTACCAGAAAGCACTACCAGCCTTACGCTCCTCCTAGAGACTTCG
CCGCCTACAGATCCAGAGTGAAGTTCAGCAGATCCGCCGACGCTCCTGCC
TATCAGCAGGGACAGAACCAGCTGTACAACGAGCTGAACCTGGGGAGAAG
AGAAGAGTACGACGTGCTGGACAAGCGGAGAGGCAGAGATCCTGAGATGG
GCGGAAAGCCCCAGCGGAGAAAGAATCCTCAAGAGGGCCTGTATAATGAG
CTGCAGAAAGACAAGATGGCCGAGGCCTACAGCGAGATCGGAATGAAGGG
CGAGCGCAGAAGAGGCAAGGGACACGATGGACTGTACCAGGGCCTGAGCA
CCGCCACCAAGGATACCTATGATGCCCTGCACATGCAGGCCCTGCCACCT
AGATGATGA

T cells may be engineered to express BAT-CARs in accordance with known techniques. Generally, a polynucleotide vector is constructed that encodes the BAT-CAR and the vector is transfected into a population of T cells. The cells are then grown under conditions promoting expression of the polynucleotide encoding the BAT-CAR by the T cells. Successful transfection (or transduction which refers to viral-mediated gene integration) and display of BAT-CARs by T cells may be conducted via standard techniques.

In some embodiments, T cells may be engineered to produce BAT-CARs by first constructing a retroviral vector encoding a selected BAT-CAR. Retroviral transduction may be performed using known techniques (e.g., Johnson et al. Blood 114:535-546 (2009)). The surface expression of BAT-CAR on transduced T cells may be determined, for example, by flow cytometry.

Populations of BAT-CAR T cells may be formulated for administration to a subject using known techniques. Formulations including populations of BAT-CAR-expressing T cells may include one or more pharmaceutically acceptable excipients. Excipients included in the formulations may have different purposes depending, for example, on the nature of the tag-binding domain, the subpopulation of T cells used, and the mode of administration. Representative examples of excipients include saline, buffered saline, dextrose, water-for-infection, glycerol, ethanol, and combinations thereof, stabilizing agents, solubilizing agents and surfactants, buffers and preservatives, tonicity agents, bulking agents, and lubricating agents. The formulations including populations of BAT-CAR T cells are typically prepared and cultured in the absence of any non-human components such as animal serum (e.g., bovine serum albumin).

A formulation may include one population of BAT-CAR T cells, or more than one, such as two, three, four, five, six or more populations of BAT-CAR-expressing T cells. The different populations of BAT-CAR T cells may differ in terms of the activation domain, the identity of the subpopulation of T cells, etc.

### Systems and Kits of the Invention

Any of the compositions described herein may be comprised in a kit or system. In a non-limiting example, one or more cells for use in cell therapy and/or the reagents to generate one or more cells for use in cell therapy that harbors recombinant expression vectors may be comprised in a kit or system. In some embodiments, a kit includes one or more of the bifunctional compounds disclosed herein. In other embodiments, a kit includes one or more of the bifunctional compounds disclosed herein and one or more reagents (e.g., genetic constructs, delivery vectors) for producing autologous CAR-T cells. In yet other embodiments, a kit includes one or more of the bifunctional compounds disclosed herein and allogeneic CAR-T cells. In any of the above-described embodiments, a kit optionally includes a ROS/RNS-generating agent. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments of the invention, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR as described herein and/or regulatory elements therefor.

In particular embodiments, there are one or more apparati in the kit suitable for extracting one or more samples from an individual. The apparatus may include a syringe, scalpel, and so forth.

In some cases of the invention, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### Methods

Methods of the present invention include treatment of subjects having or diagnosed with cancer. As used herein, the terms “treat”, “treating”, and “treatment” have their ordinary and customary meanings, and include one or more of blocking, ameliorating, or decreasing in severity and/or frequency a symptom of cancer in a subject. In some embodiments, the subject receiving treatment is a human. In other embodiments, the subject is a non-human animal, e.g., a non-human primate, bird, horse, cow, goat, sheep, a companion animal, such as a dog, cat or rodent, or other mammal.

Cancers that may be amenable to treatment with the treatment modalities of the present invention are characterized by presence of solid tumors. Broadly, they include adenomas, carcinomas, and sarcomas, both adult and pediatric alike.

Representative examples of cancers characterized by solid tumors which may be treated in accordance with the present invention include breast (including HER2+ and metastatic), colorectal, colon, esophageal, bile duct, lung (including small cell and non-small cell lung tumors, mesothelioma, adenocarcinoma of the lung and squamous carcinoma of the lung), liver, epidermoid tumors, squamous tumors such as head and neck tumors, epithelial squamous cell cancer, thyroid, cervical, ovarian, neuroendocrine tumors of the digestive system, neuroendocrine tumors, pheochromacytomas, cancer of the peritoneum, hepatoblastoma, HPCR, brain cancers (e.g., diffuse intrinsic pontine glioma, capillary hemangioblastomas, meningiomas, and cerebral metastases, gliomas, glioblastomas (glioblastoma multiforme) and neuroblastomas, and medulloblastoma, ependymoma), bladder cancer, hepatoma, endometrial or uterine carcinoma, salivary gland carcinoma, kidney or renal cancer, bone cancer, soft tissue sarcoma (including embryonal and alveolar rhabdomyosarcoma, GIST, rectal, pancreatic, prostate, gastrointestinal (gastric and stomach), alveolar soft part sarcoma and clear cell sarcoma), cholangiocarcinoma, bile cancer, gallbladder carcinoma, myeloma, vulval cancer, penile carcinoma, retinal, androgen-dependent tumors, androgen-independent tumors. Kaposi's sarcoma, synovial sarcoma, vasoactive intestinal peptide secreting tumor, CNS neoplasms, melanoma, rhabdomyosarcoma, including, EMB, RMS, ALV, Wilm's cancer, Ewing's cancer, osteosarcoma, PNT, rhabdoid, rhabdomyosarcoma, retinoblastoma, adrenal cortical cancer, adrenal cancer, and leiomyosarcoma.

Representative examples of high ROS/RNS-generating cancers that may be particularly amenable to treatment include pancreatic cancer, Hodgkin lymphoma, prostate cancer, Kaposi's sarcoma, liver cancer, breast cancer, cholangiocarcinoma, gastric cancer, glioblastoma, lung adenocarcinoma, pancreatic ductal adenocarcinoma, mammary carcinoma, carcinoma of the lung, thyroid carcinoma and sarcoma, melanoma, carcinoma of the kidney, stomach, colon, liver, pancreas and bladder, neuroblastoma, carcinoma of the prostate, ovarian carcinoma, human papilloma virus (HPV)-positive cervix carcinoma, osteogenic sarcoma. Ewing sarcoma, rhabdomyosarcoma, fibrosarcoma, chondrosarcoma, leukemia, lymphoma and neuroendocrine tumors (Bauer et al., Anticancer Res. 34:1467-1482 (2014)).

Cancers to be treated include primary tumors and secondary or metastatic tumors e.g., metastasized from lung, breast, or prostate, as well as recurrent or refractory tumors. Recurrent tumors encompass tumors that appear to be inhibited by treatment with such agents, but which recur up to five years, or even up to ten years, or longer, after treatment is discontinued. Refractory tumors are tumors that were unresponsive or resistant to treatment with one or more conventional, approved or experimental therapies for the particular tumor type.

The therapeutic methods of the present invention may be “first-line”, i.e., an initial treatment in patients who not yet undergone any anti-cancer treatment, either alone or in combination with other treatments. The therapeutic methods of the present invention may also be “second-line” in the sense that they are administered to patients who have undergone at least one prior anti-cancer treatment regimen, e.g., chemotherapy, radioimmunotherapy, toxin therapy, prodrug-activating enzyme therapy, antibody therapy, surgical therapy, immunotherapy, radiation therapy, targeted therapy or any combination thereof either alone or in combination with other treatments. In some cases, the prior therapy may have been unsuccessful or partially successful but where the patient became intolerant to the particular treatment. Methods of the present invention may also be used as an adjuvant treatment, e.g., to inhibit reoccurrence of cancer in patients with no currently detectable disease or after surgical removal of tumor.

The formulation contains the BAT-CAR T cells in a number that is effective for the treatment of the specific cancer. Thus, therapeutically effective populations of BAT-CAR T cells are administered to subjects. The number of BAT-CAR T cells administered to a subject will vary between wide limits, depending upon the location, type, and severity of the cancer, the age and condition of the individual to be treated, etc. A physician will ultimately determine appropriate dosages to be used. In general, formulations are administered that contain from about 1×10⁴ to about 1×10¹⁰ BAT-CAR T cells. In some embodiments, the formulation contains from about 1×10⁵ to about 1×10⁹ BAT-CAR T cells, from about 5×10⁵ to about 5×10⁸ BAT-CAR T cells, or from about 1×10⁶ to about 1×10⁷ BAT-CAR T cells.

The formulation of BAT-CAR T cells may be administered to a subject in need thereof in accordance with acceptable medical practice. An exemplary mode of administration is intravenous injection. Other modes include intratumoral, intradermal, subcutaneous (s.c., s.q., sub-Q, Hypo), intramuscular (i.m.), intraperitoneal (i.p.), intra-arterial, intramedullary, intracardiac, intra-articular (joint), intrasynovial (joint fluid area), intracranial (including convection-enhanced delivery), intraspinal, and intrathecal (spinal fluids). Any known device useful for parenteral injection or infusion of the formulations can be used to effect such modes of administration. Such formulations may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The bifunctional compounds and the BAT-CAR T cells are co-administered to the subject, which for purposes of the present invention includes administration during the same treatment regimen. The compounds may be administered to a subject prior to, or concurrent with, or after administration of the BAT-CAR T cells, such that the compounds will bind the target cells and that once unmasked, the BAT-CAR cells will bind the unmasked pro-antigen or tag.

Formulations containing the bifunctional compounds may be administered to a subject in an amount which is effective for treating the specific cancer. The compounds may be formulated for administration to a subject using techniques known to the skilled artisan. Formulations of the compounds may include a pharmaceutically acceptable carrier, which may be selected based on factors such as the nature of the targeting moiety, the pro-antigen, and the mode of administration. Representative examples of generally used carriers include saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

In general, the therapeutically effective amount of the bifunctional compound administered to a subject will vary between wide limits, depending upon the location, source, identity, extent and severity of the cancer, the age and condition of the individual to be treated, etc. A physician will ultimately determine appropriate dosages to be used. Typically, formulations may contain from about 0.1 mg/kg to about 100 mg/kg body weight of the compound, and in some embodiments from about 1 mg/kg to about 10 mg/kg body weight of the compound, taking into account the routes of administration, symptoms, etc. Generally, the dosage of a compound of the present application administered to a subject to treat a disease or disorder such as cancer is in the range of 0.01 to 500 mg/kg, e.g., in the range of 0.1 mg/kg to 100 mg/kg, of the subject's body weight. For example, the dosage of compound administered to a subject may be in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a subject's body weight.

The bifunctional compounds may be administered to a subject in need thereof in accordance with acceptable medical practice. An exemplary mode of administration is intravenous injection. Other modes include intratumoral, intradermal, subcutaneous (s.c., s.q., sub-Q, Hypo), intramuscular (i.m.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of the formulations can be used to effect administration of the bifunctional compound.

Activation of the pro-antigen may, in some embodiments of cancer treatment, may be achieved simply due to the elevated levels of ROS/RNS in the tumor microenvironment. However, not all tumors naturally produce elevated quantities of ROS/RNS. Enhancement of ROS/RNS in a tumor can occur through local administration of drugs or via systemic delivery. ROS/RNS-enhancing drugs also include drugs that inhibit the breakdown of ROS/RNS. (See, e.g., Xu et al., Cancer Transl. Med. 4(1):35-38 (2018); Yang et al., J. Exp. Clin. Cancer Res. 37:266-275 (2018); Dharmaraja. J. Med. Chem. 60:3221-3240) (2017); de Sá Junior et al., Oxid. Med. Cell Longev. 2017: Article ID 2467940; and Bauer, Anticancer Res. 34:1467-1482 (2014) for central role of elevated ROS in many standard chemotherapy drugs and therapeutic strategies thereof.) Thus, in some embodiments of the present invention, the methods further entail localized administration of one or more agents, at or proximate to the tumor site, so as to elevate the ROS/RNS levels in order to activate or unmask the pro-antigen. In some embodiments, quantities of ROS/RNS in the microenvironment of a tumor may be advantageously increased by radiation. The radiation may be administered in the form of an external beam, or via brachytherapy or administration of radionuclides. Representative examples of radionuclides that can increase levels of ROS/RNS when delivered to a tumor microenvironment include gallium-68, lutetium-177, carbon-11, indium-111 and yttrium-90. In other embodiments, levels of ROS/RNS may be increased by administration of lanthanide nanoparticles. Use of such nanoparticles may be advantageous in that they reduce the amount of radiation that is required to generate increased ROS/RNS levels. In some embodiments, lanthanide nanoparticles may be preferentially taken up by certain brain cells, e.g., microglial cells, resulting in increased ROS/RNS in these cells, thus making them a preferential target for the BAT-CAR-T cells.

As an alternative or in conjunction with radiation, the present methods may entail administration of an ROS/RNS-generating agent. Such agents are known in the art. See, e.g., U.S. Patent Application Publication Nos. 2014/0228290; and 2006/0235080.

In some embodiments, the ROS/RNS generating agents are inhibitors of CD44. This protein, along with splice variants thereof, are often found overexpressed on tumor and tumor-initiating cells. Representative examples of tumors expressing or overexpressing CD44 or CD44 variants include cholangiocarcinoma, gastric cancer, glioblastoma, lung adenocarcinoma, stem and stem-like cancer cells, breast cancer, pancreatic ductal adenocarcinoma and neuroendocrine tumors. CD44 functions as a cysteine/glutamine antiporter, and pumps glutamine out of the cell and pumps in cysteine, which results in intracellular production of glutathione, which aids the tumor cell in dealing with elevated ROS/RNS. In contrast to tumor cells, normal cells do not require extra glutathione due to the fact that the endogenous levels of antioxidants in normal cells are able to handle normal levels of ROS/RNS.

Administration frequencies of formulations containing populations of BAT-CAR-T cells and formulations of the compounds, and optionally the ROS/RNS generating agents will vary depending on factors that may include the disease being treated, the structure of the BAT-CAR-T cells and the compounds, and the modes of administration. Each formulation may be independently administered 4, 3, 2 or once daily, every other day, every third day, every fourth day, every fifth day, every sixth day, once weekly, every eight days, every nine days, every ten days, bi-weekly, monthly and bi-monthly. The duration of treatment will also vary, and be based for example, on the disease being treated and will be best determined by the attending physician. However, continuation of treatment is contemplated to last for a number of days, weeks, or even months.

The methods of the present application may entail independent or co-administration of the compounds, the BAT-CAR-T cells and optionally the ROS/RNS-generating agent to the subject in a single, one-time dose, or in multiple doses (e.g., 2, 3, 4, 5, 6, 7, 8, 10, 15, 20, or more doses). Thus, the frequency of co-administration may range from once up to about once every eight weeks. In another example, the frequency of administration ranges from about once a week up to about once every six weeks. In some embodiments, the frequency of administration ranges from about once every three weeks up to about once every four weeks. In other embodiments, the BAT-CAR-T cells may be administered in a single, one-time dose, while the frequency of administration of the bifunctional compounds and optionally the ROS/RNS-generating agents may range from a single dose to once every day to once every week up to about once every 4-6 weeks.

In some embodiments, the bifunctional compound and CAR-T cells are administered more than once, e.g., at least two times, at least three times, at least four times, at least five times, at least six times, at least seven times, at least eight times, at least nine times, or at least ten times. In some embodiments, the bifunctional compound, the ROS/RNS-generating agent and CAR-T cells are administered more than once, e.g., at least two times, at least three times, at least four times, at least five times, at least six times, at least seven times, at least eight times, at least nine times, or at least ten times. In some embodiments, the bifunctional compound and CAR-T cells are administered through various parenteral routes of administration, e.g., intravenously, intratumorally, intradermally, subcutaneously (s.c., s.q., sub-Q. Hypo), intramuscularly (i.m.), intraperitoneally((i.p.), intra-arterially, intramedullarilly, intracardially, intra-articularly, intrasynovially, intracranially, intraspinally, and intrathecally. In some embodiments, the bifunctional compound, the ROS/RNS-generating agent and CAR-T cells are administered through various parenteral routes of administration, e.g., intravenously, intratumorally, intradermally, subcutaneously (s.c., s.q., sub-Q. Hypo), intramuscularly (i.m.), intraperitoneally (i.p.), intra-arterially, intramedullarilly, intracardially, intra-articularly, intrasynovially, intracranially, intraspinally, and intrathecally.

### Combination Therapy

In certain embodiments, the inventive methods of treating cancer may be part of a combination therapy wherein the subject is also treated with another anti-cancer agent. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapies. In the context of the present invention, it is contemplated that cell therapy could be used similarly in conjunction with chemotherapeutic, radiotherapeutic, or immunotherapeutic intervention, as well as pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the times of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

It is expected that the treatment cycles would be repeated as necessary. It also is contemplated that various standard therapies, as well as surgical intervention, may be applied in combination with the inventive cell therapy.

### Chemotherapy

Cancer therapies also include a variety of combination therapies with both chemical and radiation based treatments. Combination chemotherapies include, for example, abraxane, altretamine, docetaxel, herceptin, methotrexate, novantrone, zoladex, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing and also combinations thereof.

In specific embodiments, chemotherapy for the individual is employed in conjunction with the invention, for example before, during and/or after administration of the invention

### Radiotherapy

Other factors that cause DNA damage and have been used extensively include what are commonly known as gamma-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include PD-1, PD-L1, CTLA4, carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

### Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

### Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor: interferon alpha, beta, and gamma; IL-2 and other cytokines: F42K and other cytokine analogs: or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

These and other aspects of the present invention will be further appreciated upon consideration of the following working example, which is intended to illustrate certain particular embodiments of the invention, but which is not intended to limit its scope, as defined by the claims.

### EXAMPLES

### Example 1: Construction of Anti-Fluorescein CAR T and its Components

Construct #1, encoding a polypeptide comprising “anti-Fluorescein CAR T”

Anti-fluorescein-4M5.3 antibody (antigen binding domain)
(SEQ ID NO: 4)
DVVMTQTPLSLPVSLGDQASISCRSSQSLVHSNGNTYLRWYLQKPGQSPKVLIYKV
SNRVSGVPDRFSGSGSGTDFTLKINRVEAEDLGVYFCSQSTHVPWTFGGGTKLEIKS
SADDAKKDAAKKDDAKKDDAKKDGGVKLDETGGGLVQPGGAMKLSCVTSGFTF
GHYWMNWVRQSPEKGLEWVAQFRNKPYNYETYYSDSVKGRFTISRDDSKSSVYL
QMNNLRVEDTGIYYCTGASYGMEYLGQGTSVTVS
Signal peptide-CD8a-sp|P01732|1-21
(SEQ ID NO: 5)
MALPVTALLLPLALLLHAARP
Hinge region-CD8a-sp|P01732|138-182
(SEQ ID NO: 6)
TTTPAPRPPTPAPTIASQPLSLRPEACRPAAGGAVHTRGLDFACD
Transmembrane region (TM)-CD28-sp|P10747|153-179
(SEQ ID NO: 7)
FWVLVVVGGVLACYSLLVTVAFIIFWV
Intracellular domain (ICD)-CD28-sp|P10747|180-220
(SEQ ID NO: 8)
RSKRSRLLHSDYMNMTPRRPGPTRKHYQPYAPPRDFAAYRS
Intracellular domain (ICD)-41BB-sp/Q07011|214-255
(SEQ ID NO: 9)
KRGRKKLLYIFKQPFMRPVQTTQEEDGCSCRFPEEEEGGCEL
Intracellular domain (ICD)-CD3z-sp|P20963|52-164
(SEQ ID NO: 10)
RVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHMQ
ALPPR
Anti-fluorescein Third Generation CAR T-full peptide
(SEQ ID NO: 11)
MALPVTALLLPLALLLHAARPDVVMTQTPLSLPVSLGDQASISCRSSQSLVHSNGNT
YLRWYLQKPGQSPKVLIYKVSNRVSGVPDRFSGSGSGTDFTLKINRVEAEDLGVYF
CSQSTHVPWTFGGGTKLEIKSSADDAKKDAAKKDDAKKDDAKKDGGVKLDETGG
GLVQPGGAMKLSCVTSGFTFGHYWMNWVRQSPEKGLEWVAQFRNKPYNYETYY
SDSVKGRFTISRDDSKSSVYLQMNNLRVEDTGIYYCTGASYGMEYLGQGTSVTVST
TTPAPRPPTPAPTIASQPLSLRPEACRPAAGGAVHTRGLDFACDFWVLVVVGGVLA
CYSLLVTVAFIIFWVRSKRSRLLHSDYMNMTPRRPGPTRKHYQPYAPPRDFAAYRS
KRGRKKLLYIFKQPFMRPVQTTQEEDGCSCRFPEEEEGGCELRVKFSRSADAPAYQ
QGQNQLYNELNLGRREEYDVLDKRRGRDPEMGGKPQRRKNPQEGLYNELQKDK
MAEAYSEIGMKGERRRGKGHDGLYQGLSTATKDTYDALHMQALPPR**
Anti-fluorescein Second Generation (41BB only) CAR T-full peptide
(SEQ ID NO: 12)
MALPVTALLLPLALLLHAARPDVVMTQTPLSLPVSLGDQASISCRSSQSLVHSNGNT
YLRWYLQKPGQSPKVLIYKVSNRVSGVPDRFSGSGSGTDFTLKINRVEAEDLGVYF
CSQSTHVPWTFGGGTKLEIKSSADDAKKDAAKKDDAKKDDAKKDGGVKLDETGG
GLVQPGGAMKLSCVTSGFTFGHYWMNWVRQSPEKGLEWVAQFRNKPYNYETYY
SDSVKGRFTISRDDSKSSVYLQMNNLRVEDTGIYYCTGASYGMEYLGQGTSVTVST
TTPAPRPPTPAPTIASQPLSLRPEACRPAAGGAVHTRGLDFACDFWVLVVVGGVLA
CYSLLVTVAFIIFWVKRGRKKLLYIFKQPFMRPVQTTQEEDGCSCRFPEEEEGGCEL
RVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHMQ
ALPPR**
Anti-fluorescein Second Generation (CD28 only) CAR T-full peptide
(SEQ ID NO: 13)
MALPVTALLLPLALLLHAARPDVVMTQTPLSLPVSLGDQASISCRSSQSLVHSNGNT
YLRWYLQKPGQSPKVLIYKVSNRVSGVPDRFSGSGSGTDFTLKINRVEAEDLGVYF
CSQSTHVPWTFGGGTKLEIKSSADDAKKDAAKKDDAKKDDAKKDGGVKLDETGG
GLVQPGGAMKLSCVTSGFTFGHYWMNWVRQSPEKGLEWVAQFRNKPYNYETYY
SDSVKGRFTISRDDSKSSVYLQMNNLRVEDTGIYYCTGASYGMEYLGQGTSVTVST
TTPAPRPPTPAPTIASQPLSLRPEACRPAAGGAVHTRGLDFACDFWVLVVVGGVLA
CYSLLVTVAFIIFWVRSKRSRLLHSDYMNMTPRRPGPTRKHYQPYAPPRDFAAYRS
RVKFSRSADAPAYQQGQNQLYNELNLGRREEYDVLDKRRGRDPEMGGKPQRRKN
PQEGLYNELQKDKMAEAYSEIGMKGERRRGKGHDGLYQGLSTATKDTYDALHMQ
ALPPR**

### Example 2: Construction of Anti-Anthraquinone-2-Carboxylate CAR T and its Components

Construct #2, encoding a polypeptide comprising “anti-anthraquinone-2-carboxylate CAR T”

Anti-anthraquinone-2-carboxylate - MC48.B11
antibody (antigen binding domain)
(SEQ ID NO: 14)
QVRLQGSGPSLVKPSQTLSLTCTVSGFSLTSNAVDWVRQAPGKVPEWLGF
IRGGGSTFYNSALKSRLSITRDTSKSQVSLSLSSVTTEDTAVYYCARASC
SGDIYTDTCGIDYWGPGLLVTVSSEGKSSGSGSESKVDQSALTQPSSVSR
SLGQSVSITCSGSSSNVGAGNYVNWFRLIPGSAPKSLIYAATTRASGVPD
RFSGSRSGNTATLTISSLQAEDEADYYCSSYDITAVNLFGSGTRLTVLG
Signal peptide - CD8a - sp|P01732|1-21
(SEQ ID NO: 5)
MALPVTALLLPLALLLHAARP
Hinge region - CD8a - sp|P01732|138-182
(SEQ ID NO: 6)
TTTPAPRPPTPAPTIASQPLSLRPEACRPAAGGAVHTRGLDFACD
Transmembrane region (TM) - CD28 - sp|P10747|
153-179
(SEQ ID NO: 7)
FWVLVVVGGVLACYSLLVTVAFIIFWV
Intracellular domain (ICD) - CD28 - sp|P10747|
180-220
(SEQ ID NO: 8)
RSKRSRLLHSDYMNMTPRRPGPTRKHYQPYAPPRDFAAYRS
Intracellular domain (ICD) - 41BB - sp|Q07011|
214-255
(SEQ ID NO: 9)
KRGRKKLLYIFKQPFMRPVQTTQEEDGCSCRFPEEEEGGCEL
Intracellular domain (ICD) - CD3z - sp|P20963|
52-164
(SEQ ID NO: 10)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR
Anti-anthraquinone-2-carboxylate CAR T - full
peptide
(SEQ ID NO: 15)
MALPVTALLLPLALLLHAARPQVRLQGSGPSLVKPSQTLSLTCTVSGFSL
TSNAVDWVRQAPGKVPEWLGFIRGGGSTFYNSALKSRLSITRDTSKSQVS
LSLSSVTTEDTAVYYCARASCSGDIYTDTCGIDYWGPGLLVTVSSEGKSS
GSGSESKVDQSALTQPSSVSRSLGQSVSITCSGSSSNVGAGNYVNWFRLI
PGSAPKSLIYAATTRASGVPDRFSGSRSGNTATLTISSLQAEDEADYYCS
SYDITAVNLFGSGTRLTVLGTTTPAPRPPTPAPTIASQPLSLRPEACRPA
AGGAVHTRGLDFACDFWVLVVVGGVLACYSLLVTVAFIIFWVRSKRSRLL
HSDYMNMTPRRPGPTRKHYQPYAPPRDFAAYRSKRGRKKLLYIFKQPFMR
PVQTTQEEDGCSCRFPEEEEGGCELRVKFSRSADAPAYQQGQNQLYNELN
LGRREEYDVLDKRRGRDPEMGGKPQRRKNPQEGLYNELQKDKMAEAYSEI
GMKGERRRGKGHDGLYQGLSTATKDTYDALHMQALPPR**

### Example 3: Characterization of Boronate-Caged Fluoroscein with Polyethylene Glycol (PEG) Linker to a Maleimide Moiety

Compound 1 was synthesized according to the method outlined in Scheme 3 with pinanediol boronate ester. (C₅₄H₆₁B₂N₃O₁₃, MW 981.70, log P 5.32)

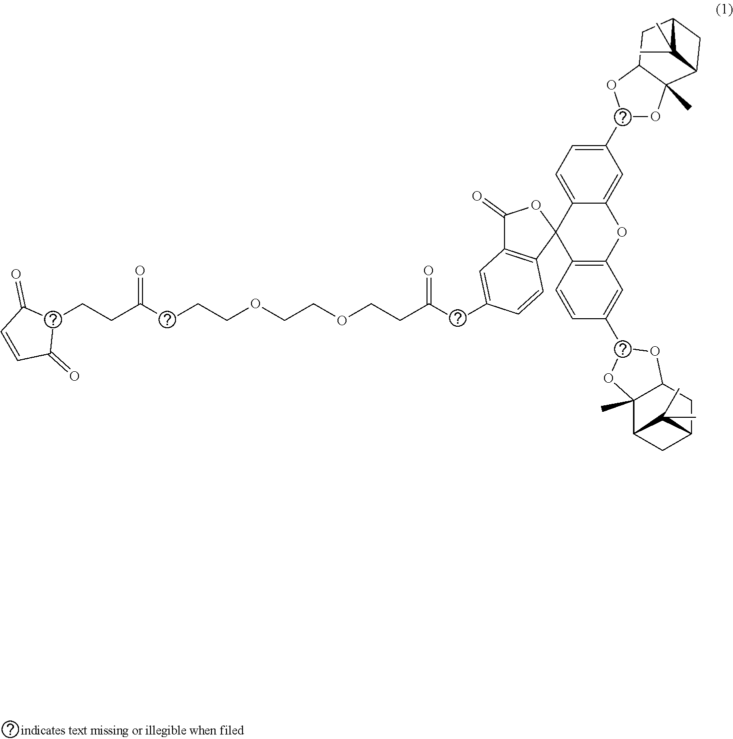 [SMILES: CC1(C)[C@@H]2CC3OC(c4ccc5c(c4)Oc4cc(C6OC7C[C@@H]8C[C@@H](C8(C)C)[C@]7(C)O6)ccc4C54OC(=O)c5cc(CC(=O)CCOCCOCCCC(=O)CCC6C(=O)C=CC6=O)ccc54)O[C@@]3(C)[C@H]1C2]

### 3-(2,5-dioxo-2,5-dihydro-1H-pyrrol-1-yl)-N-(2-(2-(3-oxo-3-((3-oxo-3′,6′-bis((3aS,4S,6S)-3a,5,5-trimethylhexahydro-4,6-methanobenzo[d][1,3,2]dioxaborol-2-yl)-3H-spiro[isobenzofuran-1,9′-xanthen]-5-yl)amino)propoxy)ethoxy)ethyl)propanamide

Compound 2 or Ex-1 was synthesized according to the method outlined in Scheme 1.(C₃₄H₃₁N₃O₁₁, MW 657.62, log P 2.86)

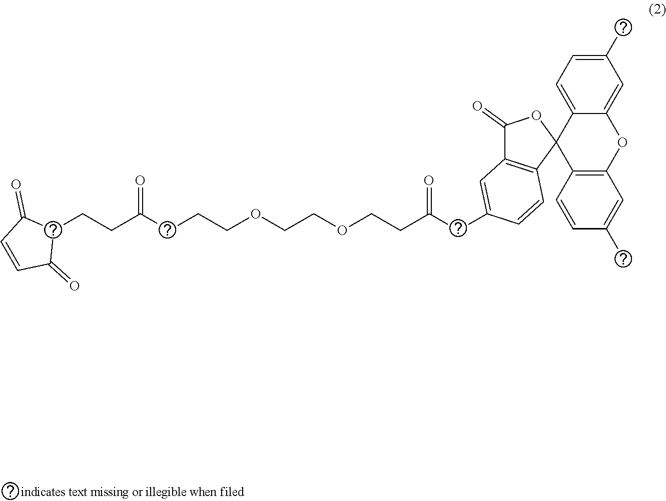 [SMILES: Cc1ccc2c(c1)Oc1cc(C)ccc1C21OC(=O)c2cc(CC(=O)CCOCCOCCCC(=O)CCC3C(=O)C=CC3=O)ccc21]

### N-(3′,6′-dihydroxy-3-oxo-3H-spiro[isobenzofuran-1,9′-xanthen]-5-yl)-3-(2-(2-(3-(2,5-dioxo-2,5-dihydro-1H-pyrrol-1-yl)propanamido)ethoxy)ethoxy)propanamide

### Stock Solutions/Solubility

Compound 1 was not soluble in buffer solution 50 mM 4-(2-hydroxyethyl)-1-piperazineethanesulfonic acid (HEPES) pH 7 at 2 mg/ml at room temperature overnight. In contrast, DMSO readily dissolved compound 1 at 2 mg/ml forming a clear solution. Compound 2 dissolved immediately at 1.5 mg/ml in buffer solution 50 mM HEPES pH 7 forming a yellow solution.

In the experiments described below, compound 1 samples were prepared by dilution of the 2 mg/ml stock in DMSO into 50 mM HEPES pH 7 buffer solution. Aqueous solutions were made at concentrations of up to 40 μM compound 1 (2% DMSO). Similarly, compound 2 solutions were prepared by dilution from the 1.5 mg/ml stock in 50 mM HEPES pH 7 buffer solution.

### Ultraviolet/Visible (UV/Vis) Spectroscopy

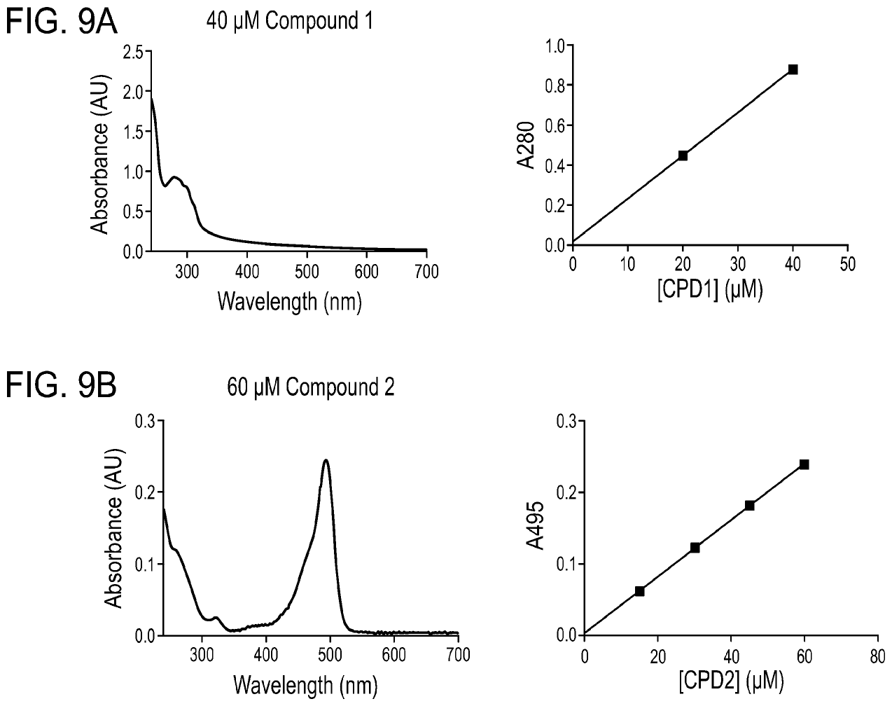

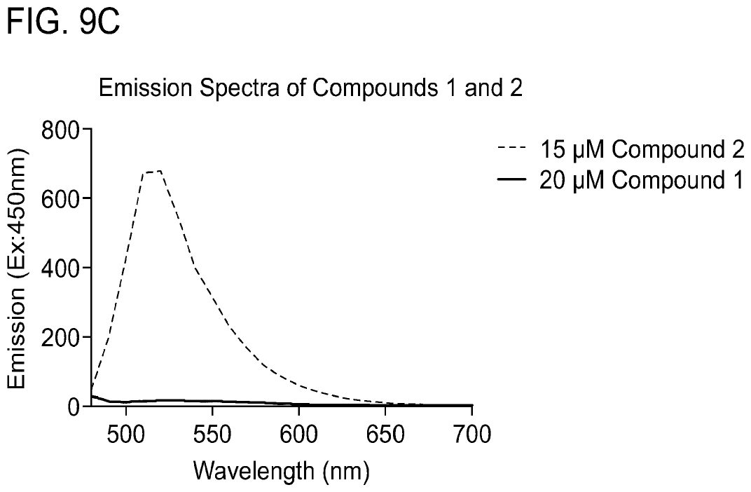

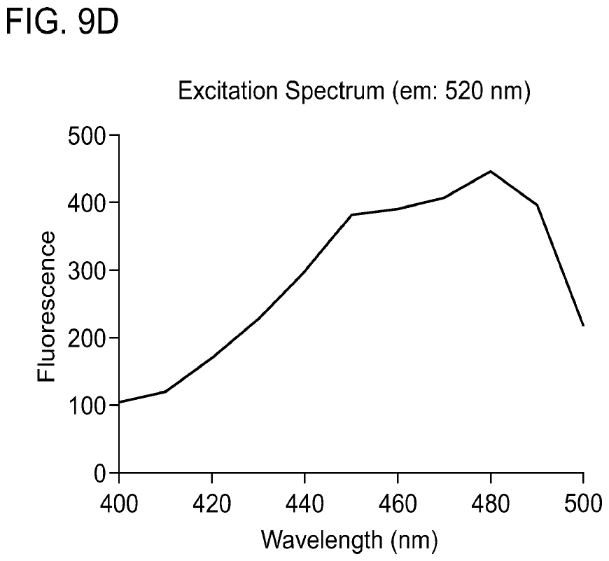

A solution of compound 1 in 250 mM HEPES pH 7 buffer solution/2% DMSO at 20 and 40 μM gave an absorbance UV/Vis spectrum with maximum at 280 nm (HP 8453 Diode Array Spectrophotometer, Agilent/HP). Absorbance at 280 nM was linear up to at least 40 μM with and extinction coefficient of 2230 M⁻¹ cm⁻¹ (FIG. 9A).

Compound 2 also had an ultraviolet absorbance with a maximum of about 280 nM. An additional visible absorption peak was observed at 495 nm. Visible absorbance at 495 nm was linear with concentration up to at least 60 μM with an extinction coefficient of 3980 M⁻¹ cm⁻¹ (FIG. 9B).

### Fluorescence Spectroscopy

Samples of compounds 1 and 2 were prepared in 50 mM HEPES pH 7 buffer solution by 1:100 dilution of the stock solutions for concentration of 20 μM compound 1 and 15 μM compound 2. Emission spectra were recorded with excitation at 450 nm and emission at 470-700 nm (10-nm increments) (Spectramax® M2e Microplate Reader, Molecular Devices LLC). The emission maximum for compound 2 was 520-530 nm in FIG. 9C.

An excitation spectrum of compound 2 was also recorded observing emission at 520 nm. Maximum fluorescence was observed at excitation 480 nm (FIG. 9D).

### Example 4: Uncaging of Compound 1 with Hydrogen Peroxide (H₂O₂)

Hydrogen peroxide is expected to uncage compound 1 to produce compound 2. An initial titration of H₂O₂ with compound 1 gave a concentration dependent increase in fluorescence (excitation 485, emission 525) over the course of 1 hour. 20 μM compound 1 samples were prepared by 1:100 dilution of stock into 50 mM HEPES pH 7 buffer and the reaction started by 1:1000 dilution of H₂O₂ solutions diluted in water to give final concentrations of 0.3, 0.03 or 0.003 percent. The concentration of the 30% stock H₂O₂ was confirmed by measuring the absorbance at 240 nm using an extinction coefficient of 43.6 M⁻¹ cm⁻¹.

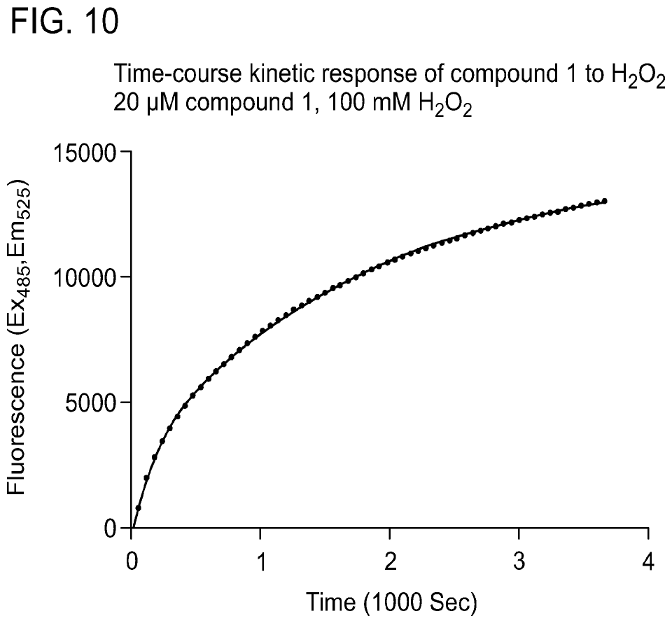

A time-course of the kinetic response of compound 1 to H₂O₂ at 100 mM H₂O₂ is illustrated in FIG. 10. Multiple (3) peaks, including compound 1, were observed by HPLC. Analysis of products suggested that the maleimide moiety is unstable in the presence of H₂O₂, at least at the relatively high tested H₂O₂ concentrations. The possible side products are shown below.

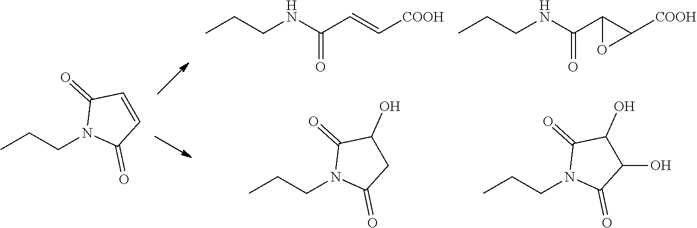

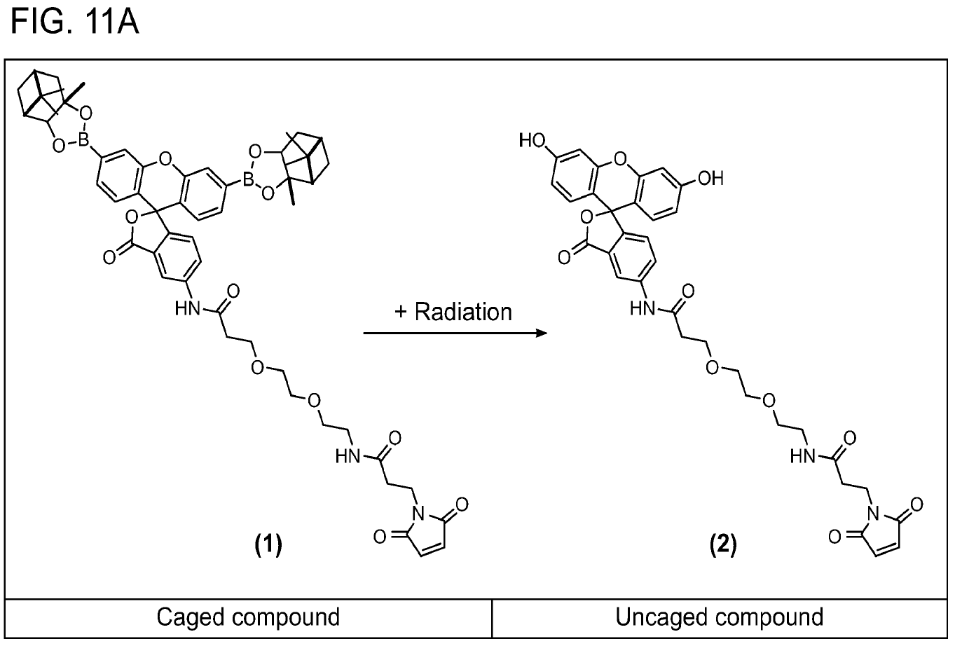

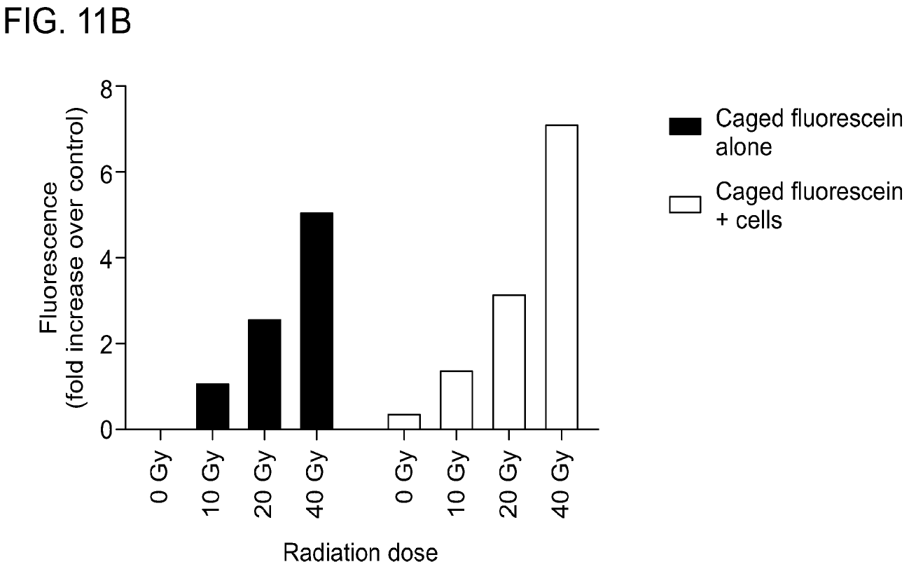

### Example 5: Uncaging of Compound 1 with Radiation (FIG. 11A)

A 20 μM solution of compound 1 in phosphate-buffered saline (PBS), 5% fetal bovine serum (FBS) was exposed to increasing radiation doses (0-40Gy) in the absence (black bars) or in the presence (gray bars) of 300,000 mouse EL4 cells in solution (FIG. 11B). The production of fluorescence was detected with a 96-well plate reader (Excitation: 485/20, Emission: 528/20). The increase in fluorescence is detected as [(fluorescence)sample-(fluorescence)control]/(fluorescence)control. The control sample is defined as a 20 μM compound 1 in PBS, 5% FBS without exposure to radiation. Each condition was tested in triplicates.

Results, illustrated in FIG. 11B, show that increasing doses of external beam radiation were used to induce the generation of ROS/RNS in a PBS, 5% FBS solution of compound 1. To simulate the catalyzing effect provided by tumor cells in the generation of ROS/RNS when exposed to radiation, the compound 1 was studied in the presence or absence of auxiliary cells. The amount of ROS/RNS generated by radiation rapidly converted compound 1 to compound 2 (FIG. 11A). The amount of compound 2 generated was proportional to the dose used and it was enhanced in the presence of cells in solution.

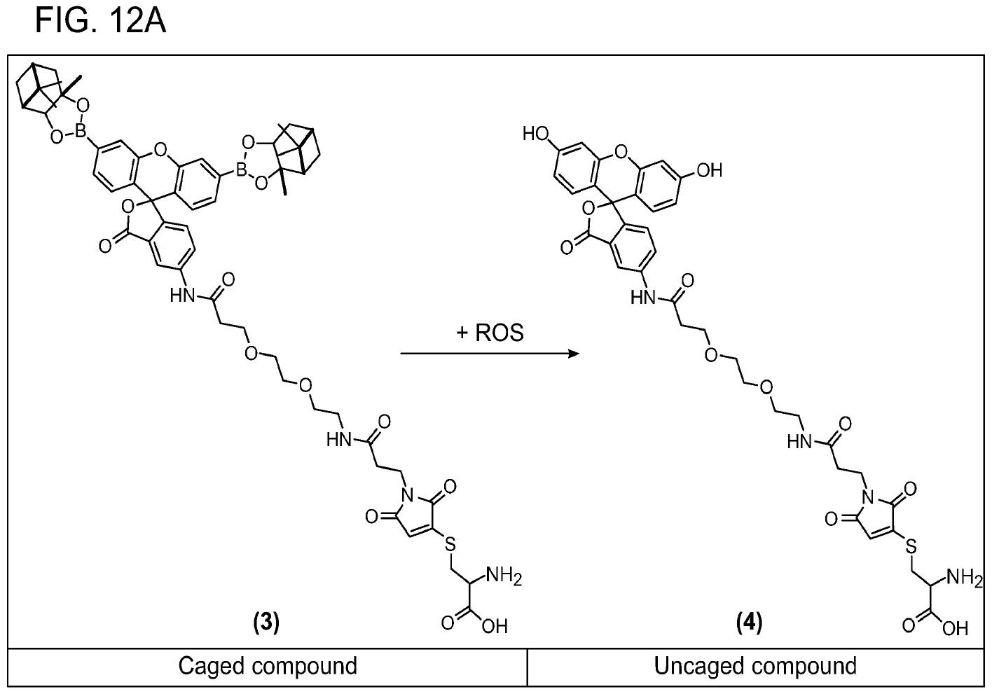

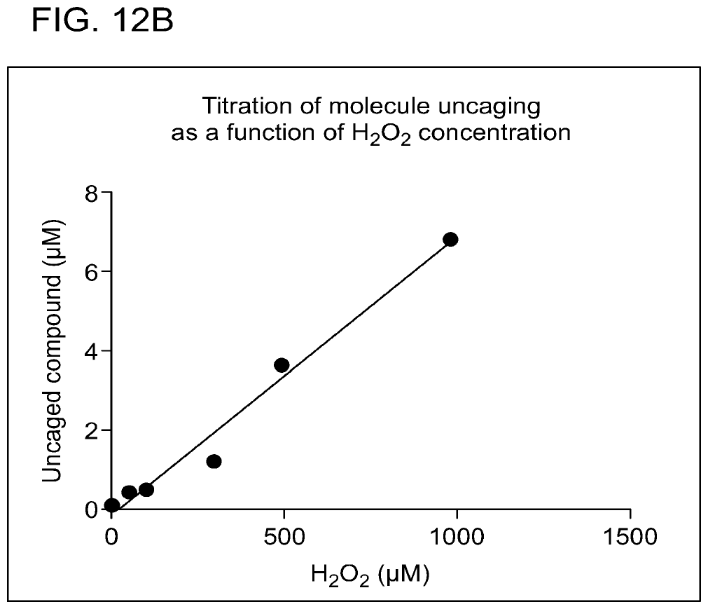

### Example 6: Uncaging of Compound 3 with Hydrogen Peroxide (H₂O₂) (FIG. 12A)

To minimalize the possibility of side reactions of the maleimide moiety in the presence of H₂O₂, compound 1 was allowed to react with cysteine to obtain Cys-adducts that may be more relevant to the antibody adducts of interests (See, e.g., Scheme 2). Cys-adducts were prepared for both compounds 1 and 2 (hereafter compound 3 and compound 4, respectively). Addition of a 10 fold excess of cysteine resulted in rapid and complete conversion to the cysteine adduct according to HPLC analysis.

A 20 μM solution of compound 3 in 50 mM HEPES pH 7 buffer was titrated with H₂O₂. Production of compound 4 was monitored by fluorescence-detected HPLC after 15 minutes reaction. Titration of compound 3 gave a linear response between 1000 and 50 μM H₂O₂ (FIG. 12B).

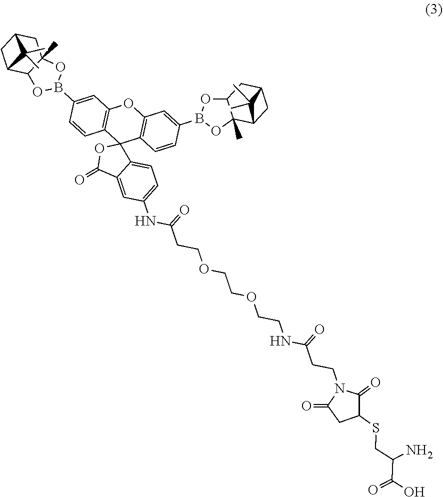

### S-(2,5-dioxo-1-(3-oxo-3-((2-(2-(3-oxo-3-((3-oxo-3′-((3aS,4S,6S)-3a,5,5-trimethylhexahydro-4,6-methanobenzo[d][1,3,2]dioxaborol-2-yl)-6′-((4R,6R,7aS)-5,5,7a-trimethylhexahydro-4,6-methanobenzo[d][1,3,2]dioxaborol-2-yl)-3H-spiro[isobenzofuran-1,9′-xanthen]-5-yl)amino)propoxy)ethoxy)ethyl)amino)propyl)pyrrolidin-3-yl)cysteine

H₂O₂ rapidly converted compound 3 to compound 4. Fluorescence-detected HPLC confirmed that H₂O₂ reaction with compound 3 produced the desired product (4). Slow uncaging of compounds 1 and 3 was observed in samples not treated with H₂O₂. Addition of 0.5M EDTA, pH 8.0 (Ultrapure Grade, Boston BioProducts) to the samples remedied the undesired uncaging of the compounds.

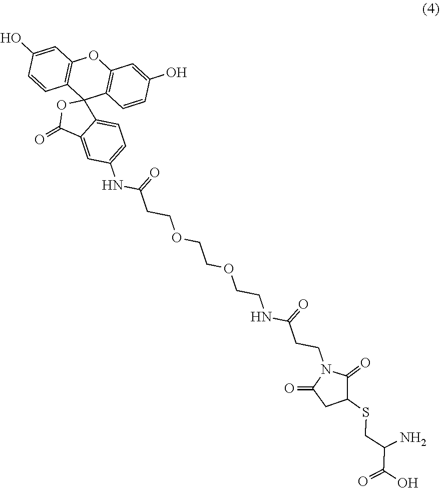 [SMILES: NC(CSC1CC(=O)N(CCC(=O)NCCOCCOCCC(=O)Nc2ccc3c(c2)C(=O)OC32c3ccc(O)cc3Oc3cc(O)ccc32)C1=O)C(=O)O]

### S-(1-(3-((2-(2-(3-((3′,6′-dihydroxy-3-oxo-3H-spiro[isobenzofuran-1,9′-xanthen]-5-yl)amino)-3-oxopropoxy)ethoxy)ethyl)amino)-3-oxopropyl)-2,5-dioxopyrrolidin-3-yl)cysteine

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-44. (canceled)

45. A composition comprising:
an immune cell comprising a chimeric antigen receptor (CAR), wherein the CAR comprises a sequence of any one of SEQ ID NO: 11-SEQ ID NO: 13.

46. The composition of claim 45, wherein the CAR comprises the sequence of SEQ ID NO: 11.

47. The composition of claim 45, wherein the CAR comprises the sequence of SEQ ID NO: 12.

48. The composition of claim 45, wherein the CAR comprises the sequence of SEQ ID NO: 13.

49. The composition of claim 45, wherein the immune cell has cytolytic activity.

50. The composition of claim 45, wherein the immune cell is a T cell.

51. The composition of claim 50, wherein the T cell is a CD3+ CD8+ T cell, a CD3+ CD4+ T cell, or a NKT cell.

52. A system comprising:
a bifunctional compound comprising a fluorescein or fluorescein derivative; and
the composition of claim 45.

53. The system of claim 52, wherein the fluorescein derivative is 5-carboxyfluorescein, 6-carboxyfluorescein, 5-(iodoacetamido)fluorescein, 5-([4,6-dichlorotriazin-2-yl]amino)fluorescein hydrochloride, 5-(bromomethyl)fluorescein, fluorescein 5(6)-isothiocyanate, or fluorescein 5-carbamoylmethylthiopropanoic acid.

54. The system of claim 52, wherein the fluorescein or fluorescein derivative is covalently linked to a targeting moiety, and wherein the targeting moiety specifically binds to a tumor antigen.

55. The system of claim 54, wherein the targeting moiety comprises an antibody, an antibody fragment, a nucleic acid, a polypeptide, a glycoprotein, a carbohydrate, or a lipid.

56. The system of claim 54, wherein the tumor antigen comprises platelet derived growth factor receptor alpha (PDGFRα), activin a receptor type 1 (ACVR1), human epidermal growth factor receptor 2 (HER2), prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, chromogranin, cytokeratin, desmin, glial fibrillary acidic protein (GFAP), gross cystic disease fluid protein (GCDFP-15), HMB-45 antigen, protein melan-A (MART-1), myo-Dl, muscle-specific actin (MSA), neurofilament, neuron-specific enolase (NSE), placental alkaline phosphatase, synaptophysis, thyroglobulin, thyroid transcription factor-1, dimeric form of pyruvate kinase isoenzyme type M2 (tumor M2-PK), an abnormal ras protein, an abnormal p53 protein, EGFRvIII, diganglioside GD2, mesothelin, interleukin 13 receptor α 2 (IL13Rα2), fibroblast activation protein (FAP), CD133, natural-killer group 2 member D (NKG2D), Ephrin type-A receptor 2 (EphA2), CD70, chondroitin sulfate proteoglycan 4 (CSPG4), CD56, CS-1, CD38, CD 138, B-cell maturation antigen (BCMA), or L1 cell adhesion molecule (L1CAM).

57. The system of claim 54, wherein the fluorescein or fluorescein derivative is covalently linked to the targeting moiety via a linker.

58. The system of claim 57, wherein the linker comprises an aliphatic linker.

59. The system of claim 57, wherein the linker comprises a polar linker.

60. The system of claim 59, wherein the polar linker comprises a polyethylene glycol moiety with 1 to 12 repeating units.

61. The system of claim 54, wherein the targeting moiety comprises a polypeptide, and the fluorescein or fluorescein derivative is covalently linked to a primary amine of the polypeptide.

62. The system of claim 54, wherein the targeting moiety comprises a polypeptide, and the fluorescein or fluorescein derivative is covalently linked to a sulfhydryl group of the polypeptide.

63. The system of claim 52, wherein the fluorescein or fluorescein derivative comprises a boronic or boronic ester derivative protecting group.

64. The system of claim 52, wherein the system comprises from 1×10⁴ to 1×10¹⁰ of the immune cell.
